(19) United States
(12) Patent Application Publication
(10) Pub. No.: US 2026/0243789 A1
(43) Pub. Date: Aug. 20, 2026

(54) SYSTEMS AND METHODS FOR TRANSPORTING A SAMPLE IN A LABORATORY

(71) Applicant: GRAIL, Inc., Menlo Park, CA (US)

(72) Inventors: David Jennions, Raleigh, NC (US); Derek Jenkins, Menlo Park, CA (US)

(73) Assignee: GRAIL, Inc., Menlo Park, CA (US)

(21) Appl. No.: 19/413,516

(22) Filed: Dec. 9, 2025

Related U.S. Application Data
(60) Provisional application No. 63/729,697, filed on Dec. 9, 2024.
(60) Provisional application No. 63/729,719, filed on Dec. 9, 2024.
(60) Provisional application No. 63/729,745, filed on Dec. 9, 2024.

(51) Int. Cl. G01N 35/00

(52) U.S. Cl. CPC G01N 35/0099

(57) ABSTRACT

In some aspects, a physical separation system configured to transport a sample between a first section of a laboratory configured to perform a first portion of a process on the sample, and a second section of the laboratory configured to perform a second portion of the process on the sample may include a chamber, and a first door positionable to optionally permit or prevent access between the first section and the chamber. The physical separation system may also include a second door positionable to optionally permit or prevent access between the second section and the chamber, and a first robotic arm configured to transport the sample between at least one of: the first section and the chamber, the second section and the chamber, or a first location within the chamber and a second location within the chamber.

### CROSS-REFERENCE TO RELATED APPLICATIONS

This application claims the benefit of priority to U.S. Provisional Patent Application No. 63/729,697, filed on Dec. 9, 2024, U.S. Provisional Patent Application No. 63/729,719, filed on Dec. 9, 2024, and U.S. Provisional Patent Application No. 63/729,745, filed on Dec. 9, 2024, all of which are incorporated herein by reference in their entireties.

### TECHNICAL FIELD

The present disclosure relates generally to the field of processing a sample in a laboratory, and in some aspects, to systems and methods for transporting a biological sample between different sections, such as a nucleic acid pre-amplification section and a nucleic acid post-amplification section of the laboratory, for processing the biological sample.

### BACKGROUND

Conventional laboratory systems often process samples through various stages, such as pre-amplification and post-amplification of nucleic acids, within the same environment. This setup can lead to cross-contamination, reducing the reliability of results. In the example of pre-amplification and post-amplification of nucleic acids, biological samples may undergo pre-amplification processes in a pre-amplification section of the laboratory, and may undergo post-amplification processes in a post-amplification section of the laboratory. The pre-amplification section of the laboratory is a different, separate section of the laboratory than the post-amplification section. Generally it is desirable to prevent cross-contamination of the biological samples in the pre-amplification section, from biological samples in the post-amplification section, and vice versa. Cross-contamination may result in erroneous data, compromised assays, or other undesirable outcomes.

A conventional laboratory may employ various manual protocols and equipment to prevent cross-contamination. For example, to prevent cross-contamination, a conventional laboratory may use protective barriers, such as laminar flow hoods, and UV sterilization. In some examples, different sections of the laboratory may be different rooms in a laboratory, with nucleic acid pre-amplification processing conducted in a first room, and nucleic acid post-amplification processing conducted in a second room, separate from the first room. The conventional laboratory may also use an environmental control system, which maintains one section of the laboratory (e.g., the pre-amplification processing section) at a relatively higher air pressure than another section of the laboratory (e.g., the post-amplification processing section). Many conventional laboratories rely on people to transfer biological samples within, as well as between or among, the separate, different sections of the laboratory.

Regardless, attempts to avoid cross-contamination between sections of the conventional laboratory have not been fully successful. Even stringent manual transfer protocols may be susceptible to human error. Further, relying on people to transfer biological samples in accordance with these stringent protocols may be time-consuming, slowing down an overall workflow within the laboratory. Still further, protective barriers and other equipment may be expensive, and may occupy large areas in the laboratory. Additionally, because traditional processes rely on people to transfer samples between separate sections of the laboratory, such traditional methods of preventing cross-contamination may not be suitable for use with a fully automated laboratory processing system.

The background description provided herein is for the purpose of generally presenting the context of the disclosure. Unless otherwise indicated herein, the materials described in this section are not prior art to the disclosure or the claims in this application and are not admitted to be prior art, or suggestions of the prior art, by inclusion in this section.

### SUMMARY OF THE DISCLOSURE

The disclosure relates to laboratory automation systems, and, more specifically, to the physical separation of components involved in different stages of laboratory processes to inhibit contamination and enhance workflow efficiency. According to one particular example, the disclosure may relate to systems and methods for transporting a biological sample between different sections, such as a pre-amplification section and a post-amplification section, of the laboratory.

In some aspects, a physical separation system configured to transport a sample between a first section of a laboratory configured to perform a first portion of a process on the sample, and a second section of the laboratory configured to perform a second portion of the process on the sample may include a chamber, and a first door positionable to optionally permit or prevent access between the first section and the chamber. The physical separation system may also include a second door positionable to optionally permit or prevent access between the second section and the chamber, and a first robotic arm configured to transport the sample between at least one of: the first section and the chamber, the second section and the chamber, or a first location within the chamber and a second location within the chamber.

In some aspects, a physical separation system may be configured to transport a sample between a first section of a laboratory configured to perform a first portion of a process on the sample, and a second section of the laboratory configured to perform a second portion of the process on the sample. The physical separation system may include at least one drawer within a wall of the laboratory, wherein the at least one drawer is positionable to optionally permit and prevent access between at least one of: the first section and the at least one drawer, or the second section and the at least one drawer. The physical separation system may also include a first robotic arm configured to transport the sample between the first section and the at least one drawer, and a second robotic arm configured to transport the sample between the second section and the at least one drawer.

In other aspects, a physical separation system configured to transport a sample between a first section of a laboratory configured to perform a first portion of a process on the sample, and a second section of the laboratory configured to perform a second portion of the process on the sample may include a pneumatic tube located within a wall of the laboratory, a first door positionable to optionally permit and prevent access between the first section and the pneumatic tube, a second door positionable to optionally permit and prevent access between the second section and the pneumatic tube, and a first robotic arm configured to transport the sample between at least one of: the first section and the pneumatic tube, or the second section and the pneumatic tube.

In still other aspects, a method of transporting a sample between a first section of a laboratory configured to perform a first portion of a process on the sample, and a second section of the laboratory configured to perform a second portion of the process on the sample via a chamber between the first section and the second section, may include picking up the sample using a robotic arm, while the sample is in the first section of the laboratory; opening a first door positionable to permit access between the first section of the laboratory and the chamber; positioning the sample, using the robotic arm, within the chamber; closing the first door to prevent access between the first section of the laboratory and the chamber; opening a second door positionable to permit access between the second section of the laboratory and the chamber; positioning the sample, using the robotic arm, within the second section of the laboratory; and closing the second door to prevent access between the second section of the laboratory and the chamber.

In further aspects, a method of transporting a sample between a first section of a laboratory configured to perform a first portion of a process on the sample, and a second section of the laboratory configured to perform a second portion of the process on the sample via a chamber between the first section and the second section, may include picking up the sample using a first robotic arm, while the sample is in the first section of the laboratory; opening a first door positionable to permit access between the first section of the laboratory and the chamber; positioning the sample, using the first robotic arm, within the chamber; closing the first door to prevent access between the first section of the laboratory and the chamber; opening a second door positionable to permit access between the second section of the laboratory and the chamber; positioning the sample, using a second robotic arm, within the second section of the laboratory; and closing the second door to prevent access between the second section of the laboratory and the chamber.

In some instances, a method of transporting a sample between a first section of a laboratory configured to perform a first portion of a process on the sample, and a second section of the laboratory configured to perform a second portion of the process on the sample via a chamber between the first section and the second section, may include picking up the sample using a first robotic arm, while the sample is in the first section of the laboratory; opening a first door positionable to permit access between the first section of the laboratory and the chamber; positioning the sample, using the first robotic arm, at a first position within the chamber; closing the first door to prevent access between the first section of the laboratory and the chamber; positioning the sample, using a second robotic arm, at a second location within the chamber; opening a second door positionable to permit access between the second section of the laboratory and the chamber; positioning the sample, using a third robotic arm, within the second section of the laboratory; and closing the second door to prevent access between the second section of the laboratory and the chamber.

In some instances, a method of transporting a sample between a first section of a laboratory configured to perform a first portion of a process on the sample, and a second section of the laboratory configured to perform a second portion of the process on the sample via a drawer between the first section and the second section, may include picking up the sample using a first robotic arm, while the sample is in the first section of the laboratory; positioning the sample, using the first robotic arm, within an interior of the drawer; isolating the first section of the laboratory from the interior of the drawer; permitting access between the interior of the drawer and the second section of the laboratory; positioning the sample, using a second robotic arm, within the second section of the laboratory; and isolating the interior of the drawer from the second section of the laboratory.

In some instances, a method of transporting a sample between a first section of a laboratory configured to perform a first portion of a process on the sample, and a second section of the laboratory configured to perform a second portion of the process on the sample via a chamber between the first section and the second section, may include picking up the sample using a first robotic arm, while the sample is in the first section of the laboratory; positioning the sample, using the first robotic arm, within an interior of a first drawer; isolating the first section of the laboratory from the interior of the first drawer; permitting access between the interior of the first drawer and the chamber; positioning the sample, using a second robotic arm, within an interior of a second drawer; isolating the chamber from the interior of the second drawer; permitting access between the interior of the second drawer and the second section of the laboratory; positioning the sample, using a third robotic arm, within the second section of the laboratory; and isolating the interior of the second drawer from the second section of the laboratory.

In some instances, a method of transporting a sample between a first section of a laboratory configured to perform a first portion of a process on the sample, and a second section of the laboratory configured to perform a second portion of the process on the sample via a pneumatic tube between the first section and the second section may include picking up the sample using a first robotic arm, while the sample is in the first section of the laboratory; positioning the sample, using the first robotic arm, within an interior of the pneumatic tube; transporting the sample through the pneumatic tube; picking up the sample, using a second robotic arm, from the pneumatic tube; and positioning the sample, using the second robotic arm, within the second section of the laboratory.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate several embodiments and together with the description, serve to explain the principles of the disclosure.

FIG. 1 depicts a schematic top view of a laboratory including multiple, different sections, as well as a physical separation system between the sections of the laboratory, according to one or more embodiments of the present disclosure.

FIG. 2 depicts a schematic top view of a portion of the laboratory of FIG. 1, with the physical separation system including a robotic arm, according to one or more embodiments of the present disclosure.

FIG. 3 depicts a schematic top view of the portion of the laboratory of FIG. 1, with the physical separation system including two robotic arms, according to one or more embodiments of the present disclosure.

FIG. 4 depicts a schematic top view of the portion of the laboratory of FIG. 1, with the physical separation system including two robotic arms and a carousel, according to one or more embodiments of the present disclosure.

FIG. 5 depicts a schematic top view of the portion of the laboratory of FIG. 1, with the physical separation system including three robotic arms and two carousels, according to one or more embodiments of the present disclosure.

FIG. 6 depicts a schematic top view of the portion of the laboratory of FIG. 1, with the physical separation system including two robotic arms and a drawer, according to one or more embodiments of the present disclosure.

FIG. 7 depicts a schematic top view of the portion of the laboratory of FIG. 1, with the physical separation system including three robotic arms and two drawers, according to one or more embodiments of the present disclosure.

FIG. 8 depicts a schematic top view of the portion of the laboratory of FIG. 1, with the physical separation system including two robotic arms and a pneumatic tube, according to one or more embodiments of the present disclosure.

FIG. 9 depicts a schematic top view of the portion of the laboratory of FIG. 1, with the physical separation system including two robotic arms and a turntable, according to one or more embodiments of the present disclosure.

FIG. 10 depicts a schematic top view of the portion of the laboratory of FIG. 1, with the physical separation system including three robotic arms and two turntables, according to one or more embodiments of the present disclosure.

FIG. 11 depicts a schematic top view of the portion of the laboratory of FIG. 1, with the physical separation system including two robotic arms and a conveyor, according to one or more embodiments of the present disclosure.

FIG. 12 depicts a schematic top view of the portion of the laboratory of FIG. 1, with the physical separation system including three robotic arms and two conveyors, according to one or more embodiments of the present disclosure.

FIG. 13 depicts a block diagram illustrating a hierarchical software architecture for an exemplary automated laboratory system in which one or more embodiments of the present disclosure may be used.

FIG. 14 depicts an exemplary computing system, according to one or more embodiments of the present disclosure.

### DETAILED DESCRIPTION OF EMBODIMENTS

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

Laboratory workflows, especially those involved in the processing and analysis of biological samples, require precision and efficiency. Conventional laboratory setups, while often labeled as autonomous, are typically only semi-autonomous. More particularly, critical steps such as sample preparation, transportation between machines, initiating specific processes, and attending to system errors still depend heavily on human intervention. This dependency introduces variability and potential errors, adversely impacting the efficiency and accuracy of the workflows. Moreover, these systems are difficult to scale without a proportionate increase in workforce and space requirements, making it challenging to meet higher throughput demands. There is a need for a truly autonomous system that can handle the variability and complexity of laboratory workflows from start to finish with little, if any, human involvement.

Conventional efforts to automate laboratory workflows have primarily focused on either automating discrete steps of the overall process or fully centralizing control within a single system. With respect to the former, step-specific automation may involve automating individual steps of the laboratory workflow rather than the entire process. More particularly, certain machines in the overall workflow may be configured to perform specific tasks, such as sample preparation, analysis, or data collection. Examples of step-specific automation may include liquid handling robots that automate pipetting and reagent dispensing, automated PCR systems that perform polymerase chain reactions with minimal human intervention, and robotic sample storage systems that automatically store and retrieve samples from refrigerated units.

However, the advantages provided by these step-specific automation processes do not address the need for a seamless, end-to-end workflow. More particularly, human operators are still required to transport samples between machines, initiate processes, and handle exceptions. As described above, human operators may be needed to transport samples between portions of a laboratory that are purposefully separated, e.g., to inhibit contamination of samples. This type of fragmented automation introduces inconsistencies and potential errors, compromising the overall efficiency and accuracy of the workflow. Additionally, the throughput of step-specific automation systems is constrained by the need for manual handling between steps, and scaling these systems requires proportionally increasing the number of human operators. Furthermore, maintenance and downtime are additional issues, as individual machines require regular maintenance and calibration, leading to disruptions in the workflow and reduced productivity or redundancy of the individual machines can be added at great additional cost and physical space.

With respect to the latter of the two conventional solutions, centralized automation systems are designed to manage all aspects of the laboratory workflow through a single, overarching controller. These systems integrate various automated machines and instruments to perform specific tasks within the workflow. Some components of centralized systems may include automated liquid handling systems for tasks such as pipetting and reagent addition, and integrated software platforms that provide a unified interface for controlling all automated components.

Despite their promise, centralized automation systems face significant issues. For instance, one of the primary problems is computational overload. More particularly, centralized systems require a single controller to manage numerous variables, including machine status, sample location, and timing for each process step, to name a few. Attempts to harmoniously manage these variables has conventionally led to computational overload, thereby causing delays, inefficiencies, and frequent system failures. Additionally, these systems still depend heavily on human operators to prepare samples, initiate processes, troubleshoot issues, and transfer samples. This reliance on human intervention introduces variability and potential errors, reducing overall efficiency and accuracy.

Scalability is another challenge encountered by centralized systems. Specifically, scaling these systems to handle higher throughput (e.g., population-level sample analysis) involves adding more automated machines, which increases the use of and reliance on the central controller, and results in computational overload. This scaling approach is limited by the controller's ability to manage additional components and the increased space required for additional machinery. Integration and compatibility issues also arise when integrating automated components from different vendors, making seamless communication and coordination between diverse machines and instruments complex and prone to errors. Lastly, centralized systems typically require complete shutdowns to conduct maintenance, upgrades, or repairs, leading to significant workflow disruptions and productivity loss.

Additionally to the foregoing, some industries, such as the automotive sector, have successfully implemented end-to-end automation, benefitting from the standardized and repetitive nature of their processes. In these industries, automation systems are designed to handle a relatively narrow range of tasks with consistent inputs and outputs, allowing for efficient and predictable operations. In contrast, laboratory workflows present a unique challenge due to their inherent variability. More particularly, laboratories often deal with a wide range of sample characteristics, sample quantities, sample types, processing methods, and analytical requirements, making it difficult to standardize workflows in the same way as the manufacturing industries. This variability introduces complexity in automation, as systems must be flexible enough to adapt to different protocols and procedures or respond to variations in a given sample including its reactions to the components of an assay. As a result, fully automating laboratory workflows requires advanced systems capable of dynamic decision-making and adaptability, which are often more challenging to develop and implement than the relatively straightforward automation systems used in industries with less variability.

In laboratory settings involving the analysis of samples, it may be important to maintain strict separation between different stages of sample processing to prevent cross-contamination. In a laboratory in which biological samples undergo different processes in separate, different sections of the laboratory, such as a pre-amplification section and a post-amplification section, generally it is desirable to prevent cross-contamination of the biological samples in one section of the laboratory from biological samples in another section of the laboratory and the general environments of the two sections. For example, in a laboratory in which biological samples undergo a first set of processes, such as pre-amplification processes (e.g., preparation and/or initial analysis of the biological samples) in a first section of the laboratory, and a second, different set of processes such as post-amplification processes (e.g., further analysis and final readouts) in a separate, second section of the laboratory, generally it is desirable to prevent contamination of the first set of biological samples undergoing the first set of processes from the second set of biological samples undergoing the second set of processes, and vice versa. Particularly when DNA amplification techniques are used, the amount of genetic material available in a post-amplification portion of the laboratory, and thus available to increase the risk of cross-contamination, is significantly greater. Cross-contamination may result in erroneous data, compromised assays, or other undesirable outcomes. Particularly, in fields such as clinical diagnostics, provision of accurate results may be of increased importance.

Conventionally, a laboratory that includes different sections for performing different processes on biological samples employs various manual protocols and equipment to prevent cross-contamination. For example, to prevent cross-contamination, a conventional laboratory may use protective barriers, such as laminar flow hoods, and UV sterilization. The conventional laboratory may also use an environmental control system, which maintains one section of the laboratory (e.g., the pre-amplification processing section) at a relatively higher air pressure than another section of the laboratory (e.g., the post-amplification processing section). In some embodiments, the pre-amplification processing section is maintained at a positive air pressure, and/or the post-amplification processing section is maintained at a negative air pressure. As a result, any contamination from one section of the laboratory to another section is contamination of biological samples in the post-amplification processing section by biological samples in the pre-amplification processing section. Generally, this contamination is less of a concern than contamination of the pre-amplification processing section by the post-amplification processing section as the amount of genetic material that may be introduced by the pre-amplification processing section is relatively lower.

Some conventional laboratories alternatively or additionally include various mechanical or pneumatic systems to transfer biological samples within and between or among the separate, different sections of the laboratory. Conventional laboratories rely on humans to transfer samples from one piece of laboratory equipment to another, or from one room to another.

Regardless, attempts to avoid cross-contamination between sections of the conventional laboratory have not been fully successful. Even stringent manual transfer protocols may be susceptible to human error. Further, transferring biological samples in accordance with these stringent protocols may be time-consuming, slowing down an overall workflow within the laboratory. Still further, protective barriers and other equipment may be expensive, and may occupy large portions of the sections. Additionally, automated systems with multiple, isolated modules may be complex to manage and/or maintain, and may often require significant downtime for cleaning and reconfiguration.

Thus, in accordance with some embodiments, the concepts described herein provide a laboratory including a physical separation system between different sections of the laboratory, such as but not limited to, a pre-amplification processing section and a post-amplification processing section of the laboratory, which may autonomously transport samples between or among the laboratory sections while avoiding contamination of the biological samples. Except to the extent as further described herein, a physical barrier, such as but not limited to a wall, otherwise may separate the different sections of the laboratory from one another, such as the pre-amplification processing section and the post-amplification processing section of the laboratory. The physical barrier may include one or more sealable openings in the wall, through which biological samples may be transported autonomously between and/or among the different sections of the laboratory.

The openings in the physical barrier (e.g., the wall) may facilitate the automated transfer of samples between the separate, different sections of the laboratory. In some embodiments, the physical separation system may include one or more robotic arms to transport biological samples within the sections, as well between the sections. In some embodiments, the robotic arm may be disposed within a chamber of the physical separation system, e.g., within the wall of the system, with the robotic arm transporting the biological sample between a first section of the laboratory and the chamber, and between a second section of the laboratory and the chamber.

In some embodiments, the physical separation system may include two robotic arms. In some embodiments, a first robotic arm (e.g., a base of the first robotic arm) may be disposed in the first section of the laboratory, and a second robotic arm (e.g., a base of the second robotic arm) may be disposed in the second section of the laboratory, the first robotic arm transporting the biological sample between the first section of the laboratory and the chamber, and the second robotic arms transporting the biological sample between the second section of the laboratory and the chamber. In other embodiments involving two robotic arms, the first and the second robotic arm may be disposed in the chamber, the first robotic arm transporting the biological sample between the first section of the laboratory and the chamber, and the second robotic arms transporting the biological sample between the second section of the laboratory and the chamber. The benefit of multiple robotic arms may be that no one robotic arm interacts with both sections of the laboratory, further promoting isolation between the two laboratory sections. In some embodiments, the physical separation system may include at least one carousel, turntable, or conveyor within the chamber, the first robotic arm transporting the biological sample between the first section of the laboratory and the turntable, carousel, or conveyor within the chamber, and the second robotic arms transporting the biological sample between the second section of the laboratory and the turntable, carousel, or conveyor within the chamber. In some embodiments, the physical separation system may include a pneumatic tube, the first robotic arm transporting the biological sample between the first section of the laboratory and the pneumatic tube, and the second robotic arms transporting the biological sample between the second section of the laboratory and the pneumatic tube. In some embodiments, the physical separation system may include at least one drawer, the first robotic arm transporting the biological sample between the first section of the laboratory and the drawer, and the second robotic arms transporting the biological sample between the second section of the laboratory and the drawer. The use of a carousel, turntable, conveyor, drawer, pneumatic tube, etc., may allow samples to be transferred between laboratory sections without direct contact between the robotic arms that pass into one of the sections of the laboratory.

In some embodiments, the physical separation system may include three or more robotic arms. In some embodiments, the first robotic arm may be disposed in the first section of the laboratory, the second robotic arm may be disposed in the second section of the laboratory, and a third robotic arm may be disposed in the chamber; the first robotic arm transporting the biological sample between the first section of the laboratory and the chamber, the second robotic arm transporting the biological sample between the second section of the laboratory and the chamber, and the third robotic arm transporting the biological sample from the first robotic arm to the second robotic arm within the chamber. In some embodiments, all three robotic arms may be disposed in the chamber; the first robotic arm transporting the biological sample between the first section of the laboratory and the chamber, the second robotic arm transporting the biological sample between the second section of the laboratory and the chamber, and the third robotic arm transporting the biological sample from the first robotic arm to the second robotic arm within the chamber. In some embodiments, the physical separation system may include at least two carousels, turntables, or conveyors within the chamber, the first robotic arm transporting the biological sample between the first section of the laboratory and the first turntable, carousel, or conveyor within the chamber, the second robotic arm transporting the biological sample between the second section of the laboratory and the second turntable, carousel, or conveyor within the chamber, and the third robotic arm transporting the biological sample between the first turntable, carousel, or conveyor and the second turntable, carousel, or conveyor. In some embodiments, the physical separation system may include at least two drawers, the first robotic arm transporting the biological sample between the first section of the laboratory and a first drawer, the second robotic arm transporting the biological sample between the second section of the laboratory and a second drawer, and the third robotic arm transporting the biological sample between the first drawer and the second drawer.

In some embodiments, the first section and the second section of the laboratory remain isolated from one another prior to transfer of the biological sample between the first section and the second section. For example, doors or windows to the chamber, the pneumatic tube, and/or the drawer or drawers may remain closed and may act as an airlock. When transporting the biological sample between the first section and the chamber, the pneumatic tube, and/or the drawer or drawers, the chamber, the pneumatic tube, and/or the drawer or drawers remain closed to the second section. Further, when transporting the biological sample between the second section and the chamber, the pneumatic tube, and/or the drawer or drawers, then the chamber, the pneumatic tube, and/or the drawer or drawers may remain closed to the first section. In some aspects, embodiments described herein may be used in conjunction with environmental controls, such as pressure differentials between the different sections of the laboratory. For example, one section of the laboratory (e.g., the pre-amplification processing section) may be maintained at a relatively higher air pressure than another section of the laboratory (e.g., the post-amplification processing section). In some embodiments, the pre-amplification processing section may be maintained at a positive air pressure, and/or the post-amplification processing section may be maintained at a negative air pressure.

Further, the chamber, the pneumatic tube, the drawer or drawers, and/or the biological sample may be sanitized or sterilized at any time before, during, or after transporting. In some aspects, one or more components of the transport system and/or the chamber may be sanitized or sterilized regularly to inhibit or avoid cross-contamination. Accordingly, cross-contamination of one of the sections of the laboratory from another section of the laboratory may be inhibited or avoided. Further, the use of the robotic arm or arms, chamber with doors or windows, turntable or turntables, carousel or carousels, conveyor or conveyors, drawer or drawers, and/or pneumatic tube, and the like, may be automated partially or fully, thereby reducing or avoiding human error that would otherwise result in cross-contamination of one or more sections of the laboratory.

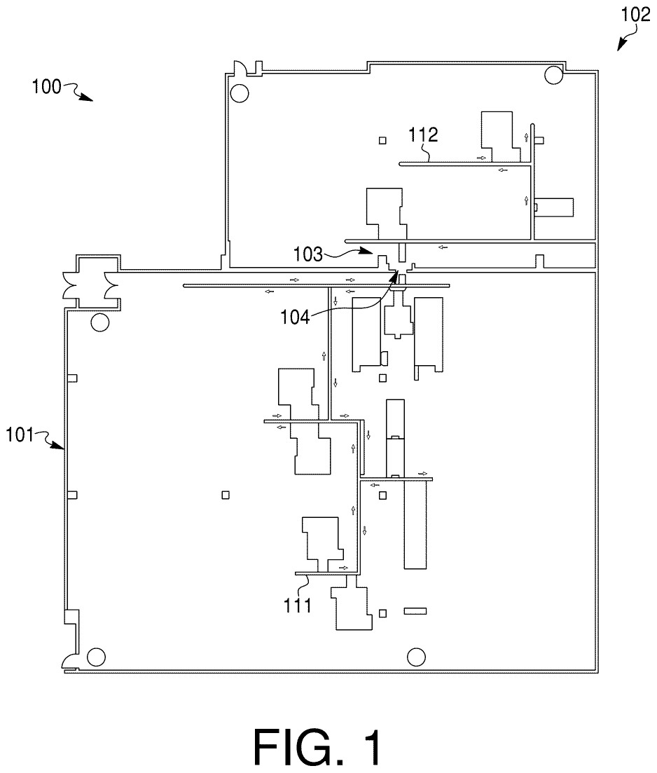

With reference to the drawings, FIG. 1 depicts a schematic top view of an exemplary laboratory including multiple, different sections, as well as a physical separation system between the sections of the laboratory, according to one or more embodiments of the present disclosure. For example, the different sections of the laboratory may include one or more pre-amplification processing sections, in which pre-amplification processing including one or more pre-amplification processes are performed on one or more biological samples. By way of further example, the different sections of the laboratory may include one or more post-amplification processing sections, in which post-amplification processing including one or more post-amplification processes are performed on one or more biological samples. The post-amplification processing may or may not occur on the biological sample on which the pre-amplification processing was performed. In some embodiments, one or more of the different sections of the laboratory may be isolated from one another, except to the extent that the physical separation system transports one or more of the biological samples between or among the different sections of the laboratory, as further described.

With reference to FIG. 1, as the drawing illustrates, a laboratory 100 may include a first section 101 and a second section 102, with a physical separation system 103 between the first section 101 and the second section 102 of the laboratory 100. Although FIG. 1 shows the laboratory 100 including the first section 101 and the second section 102, it is understood that the laboratory 100 is not limited only to two sections. For example, the laboratory 100 may include three sections, four sections, five sections, or more than five sections, such as but not limited to one or more of the first sections 101, one or more of the second sections 102, and/or one or more other sections. In some embodiments, as FIG. 1 illustrates, when the laboratory 100 includes the first section 101 and the second section 102, the biological sample may move throughout the first section 101 by a first conveyance platform 111, and/or the biological sample may move throughout the second section 102 by a second conveyance platform 112. The first and second conveyance platforms 111, 112 may include one or more suitable hardware components, such as tracks, belts, grippers, pucks, drones, robotics, etc. As shown, the first conveyance platform 111 may be entirely within the first section 101 of the laboratory 100, and the second conveyance platform 112 may be entirely within the second section 102, so that transportation of the biological sample between the first section 101 and the second section 102 occurs by way of the physical separation system 103. It is understood that either or both of the first section 101 and/or the second section 102 may omit its conveyance platform, and/or may include multiple conveyance platforms. In some embodiments, when the laboratory 100 includes more than two sections, each of the sections may or may not include a conveyance platform, and the conveyance platform may or may not be entirely within the section, such that transportation of the biological sample between or among the sections occurs by way of one or more physical separation systems.

The first section 101 of the laboratory 100 may be a pre-amplification processing section, in which one or more pre-amplification processes are performed on the biological sample. Thus, the first conveyance platform 111 may transport the biological sample between and/or among different stations within the first section 101 of the laboratory 100 at which one or more pre-amplification processes are performed on the biological sample, and may transport the biological sample to and/or from a location within the first section 101 which is proximate the physical separation system 103. The second section 102 of the laboratory 100 may be a post-amplification processing section, in which one or more post-amplification processes are performed on the biological sample. Thus, the second conveyance platform 112 may transport the biological sample between and/or among different stations within the second section 102 at which one or more pre-amplification processes are performed on the biological sample, and may transport the biological sample to and/or from a location within the second section 102 which is proximate the physical separation system 103. It is contemplated, but it is not required, that the biological sample on which the post-amplification processing occurs in the second section 102 of the laboratory 100 may have been subject to pre-amplification processing in the first section 101 of the laboratory 100.

Although FIG. 1 illustrates the laboratory 100 including the first section 101 and the second section 102, and the foregoing describes that in some embodiments the first section 101 is a pre-amplification processing section and that the second section 102 is a post-amplification processing section, the laboratory 100 may include one or more pre-amplification sections, one or more one post-amplification sections, and/or one or more other sections. Further, although embodiments of the disclosure discuss the handling of biological samples, and, more particularly, pre-amplification sections and post-amplification sections, the physical separation systems described herein may be used in any suitable industry. For example, samples may include biological samples (e.g., blood samples, urine samples, saliva samples, stool samples, cerebrospinal fluid samples, pleural fluid samples, interstitial fluid samples, tissue samples, etc.), environmental samples (e.g., water samples, soil samples, etc.), food and beverage samples, forensic samples (e.g., crime scene samples, toxicology samples, etc.), industrial samples (e.g., pharmaceutical samples, chemical samples, etc.), and the like. While pre-and post-amplification are described as exemplary processes performed in a laboratory that may require inhibition of cross-contamination and physical separation, systems of the disclosure may be used with any processes that require inhibition of cross-contamination and physical separation in any industry.

The physical separation system 103 may transport the biological sample between and/or among the different sections of the laboratory 100, such as but not limited to between the first section 101 and the second section 102 of the laboratory 100. For example, the physical separation system 103 may transport the biological sample, which has undergone pre-amplification processing in the first section 101 of the laboratory 100, from the first section 101 to the second section 102 of the laboratory 100, so that the biological sample may undergo subsequently post-amplification processing in the second section 102. However, the physical separation system 103 also or alternatively may transport the biological sample from the second section 102 to the first section 101 of the laboratory 100 or vice versa, whether or not the biological sample has undergone any processing, including any pre-amplification processing such as in the first section 101 and/or any post-amplification processing such as in the second section 102.

As stated, the biological sample may include any suitable liquid and/or solid biological sample that may be used for sequencing. In some embodiments, the biological sample may be cell-based, for example, one or more types of tissue. In some embodiments, the biological sample may include one or more cell-free nucleic acid fragments. Examples of the biological sample include but are not limited to a blood sample (e.g., a DNA samples, such as a cell-free DNA (cfDNA) sample, an RNA sample, such as a cell-free RNA (cfRNA) sample, a protein sample, a serum sample, a plasma sample, and/or a whole blood sample), a urine sample, a fecal sample, a saliva sample, an interstitial fluid sample, a pleural sample, a cerebrospinal fluid sample, a tissue sample, a bone marrow sample, and/or another sample. Further, although sequencing of DNA from the biological sample is discussed herein, RNA or protein from the biological sample may alternatively or additionally be sequenced or analyzed. The biological sample may be handled within the laboratory in a variety of formats. For example, the biological sample may be contained with an individual sample tube that is manipulated as described herein. As another example, the biological sample may be contained within an individual sample tube that is collected within a sample carrier holding a plurality of sample tubes that is manipulated as described within. As another example, the biological sample may be contained within a well of a multiwell plate (e.g., a 96-well plate, although other suitable size plates are contemplated). The multiwell plate may be manipulated as described within.

Examples of sequencing data may include but are not limited to sequence read data of targeted genomic locations, partial or whole genome sequencing data of the genome represented by nucleic acid fragments in a cell-free or cell-based sample, partial or whole genome sequencing data including one or more types of epigenetic modifications (e.g., methylation), and/or combinations thereof.

In some embodiments, the first section 101 and the second section 102 of the laboratory 100 may be physically separated or isolated from one another, except to the extent that the physical separation system 103 transports the biological sample between the first section 101 and the second section 102. When the physical separation system 103 transports the biological sample between the first section 101 and the second section 102 of the laboratory 100, or prior to transporting the biological sample between the first section 101 and the second section 102, an exterior of the biological sample may be sanitized and/or sterilized, thereby inhibiting cross-contamination between the first section 101 and the second section 102 of the laboratory 100. In some embodiments, inhibiting cross-contamination of the first section 101 of the laboratory 100 by material from the second section 102 may be more important than inhibiting cross-contamination of the second section 102 of the laboratory 100 by material from the first section 101. In some embodiments, however, reducing cross-contamination of the second section 102 of the laboratory 100 by material from the first section 101 may be more important than reducing cross-contamination of the first section 101 of the laboratory 100 by material from the second section 102. In some embodiments, it is equally important to prevent cross-contamination of each of the first section 101 and the second section 102 of the laboratory 100. In some embodiments, cross-contamination may occur because materials from one of the sections of the laboratory 100 is transported to another section of the laboratory 100 by air flow therebetween.

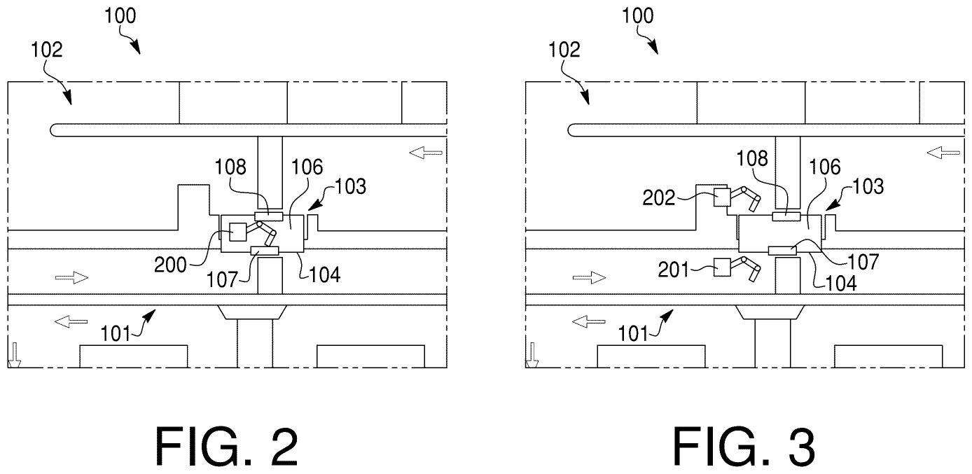

FIG. 2 depicts a schematic top view of a portion of the laboratory of FIG. 1, with the physical separation system including a robotic arm, according to one or more embodiments of the present disclosure. As FIG. 2 illustrates, the physical separation system 103 may include a robotic arm 200 that is disposed between the first section 101 and the second section 102 of the laboratory 100. In some embodiments, the robotic arm 200 may be used to transport the biological sample between the first section 101 and the second section 102 of the laboratory 100.

In some embodiments, as FIG. 2 illustrates, the physical separation system 103 may include an opening, gap, or space, referred to herein as an opening 104 within a wall between the first section 101 and the second section 102 of the laboratory 100. A chamber 106 may be disposed in the opening 104. The chamber 106 may be a volume that may be opened and closed to permit and prevent communication or exposure with one or more sections of the laboratory, such as the first section 101 and/or the second section 102 of the laboratory 100. The chamber 106 may include a first door 107, which may be optionally positionable, such as by opening and closing, to permit and prevent communication or exposure between the chamber 106 and the first section 101 of the laboratory 100. The chamber 106 may include a second door 108, which may be optionally positionable, such as by opening and closing, to permit and prevent communication or exposure between the chamber 106 and the second section 102 of the laboratory 100. The robotic arm 200 (e.g., a base of the robotic arm 200) may be disposed within the chamber 106 of the physical separation system 103. It is understood that opening and closing the first door 107 and/or the second door 108 isolates one or more of the first section 101, the second section 102, and/or the chamber 106, from one another.

The chamber 106 may include a sanitization or sterilization component that is configured to sanitize and/or sterilize items within the chamber. The sanitization or sterilization component may be used on objects or materials that are or are not only temporarily within the chamber, e.g., the robotic arm 200. The chamber 106 may include a ventilation system that is configured to vent the chamber to the first section 101 of the laboratory 100, the second section 102 of the laboratory, to an environment external to the laboratory, or to another environment. The ventilation system may be selectably operable to vent to one or more the described environments based on the present configuration of the first door 107 and second door 108. As an example, one ventilation scheme may be configured such that when the first door 107 is open and the second door 108 is closed, the chamber 106 may vent to the first section 101 of the laboratory. When the first door 107 is closed and the second door 108 is open, the chamber 106 may vent to the second section 102 of the laboratory. When the first door 107 is closed and the second door 108 is closed, the chamber 106 may vent to an external or controlled environment that is not connected to the first section 101 or the second section 102 of the laboratory. Other ventilation schemes, and other sanitization or sterilization schemes, compatible with this description are further contemplated.

In some embodiments, to transport the biological sample from the first section 101 to the second section 102 of the laboratory 100, the first door 107 of the chamber 106 may open, while the second door 108 remains closed, and at least a portion of the robotic arm 200 may extend through the open first door 107 into the first section 101. The robotic arm 200 may pick up the biological sample from the first section 101. In some examples, the robotic arm 200 may pick up the biological sample from the first conveyance platform 111. The robotic arm 200 may transport the biological sample from the first section 101 to the chamber 106. The first door 107 may close, while the second door 108 remains closed, thereby isolating the chamber 106 from each of the first section 101 and the second section 102 of the laboratory 100, and preventing cross-contamination between the first section 101 and the second section 102 of the laboratory 100. In some embodiments, the exterior of the biological sample may be sanitized and/or sterilized within the chamber 106. In some aspects, a portion of the robotic arm 200 that contacted the biological sample may also be sanitized and/or sterilized. The second door 108 of the chamber 106 may open, while the first door 107 remains closed. At least a portion of the robotic arm 200 holding the biological sample may extend through the open second door 108 into the second section 102 of the laboratory 100, transporting the biological sample from the chamber 106 to the second section 102. The robotic arm 200 may put down the biological sample in the second section 102. In some examples, the robotic arm 200 may place the biological sample on or within reach of the second conveyance platform 112. The robotic arm 200 may return to the chamber 106, such that the entire robotic arm 200 is within the chamber 106. The second door 108 of the chamber 106 may close, while the first door 107 remains closed. In some aspects, a portion of the robotic arm 200 that contacted the biological sample may be sanitized and/or sterilized at this point.

In some embodiments, the robotic arm 200 may put down and pick up the biological sample in the chamber 106, between transporting the biological sample to and from the chamber 106. In some embodiments, the robotic arm 200 may not put down and pick up the biological sample in the chamber 106, between transporting the biological sample to and from the chamber 106, but instead may hold the biological sample from when the biological sample is picked up in the first section 101 of the laboratory 100 until the biological sample is put down in the second section 102. The first conveyance platform 111 and/or the second conveyance platform 112 may or may not be used to transport the biological sample within the first section 101 and/or the second section 102 of the laboratory 100 to or from the physical separation system 103.

In some embodiments, to transport the biological sample from the second section 102 to the first section 101 of the laboratory 100, if desired, the second door 108 of the chamber 106 may open while the first door 107 remains closed, and at least a portion of the robotic arm 200 in the chamber 106 may extend from the chamber 106 through the open second door 108 into the second section 102. The robotic arm 200 may pick the biological sample from the second section 102 of the laboratory 100, and the robotic arm 200 may transport the biological sample from the second section 102 to the chamber 106. In some examples, the robotic arm 200 may pick up the biological sample from the second conveyance platform 112. The second door 108 of the chamber 106 may close, while the first door 107 remains closed, thereby isolating the chamber 106 from each of the first section 101 and the second section 102 of the laboratory 100. In some embodiments, the exterior of the biological sample may be sanitized and/or sterilized within the chamber 106. In some aspects, a portion of the robotic arm 200 that contacted the biological sample may also be sanitized and/or sterilized. The first door 107 of the chamber 106 may open, while the second door 108 remains closed. At least a portion of the robotic arm 200 holding the biological sample may extend from the chamber 106 through the open first door 107 into the first section 101 of the laboratory 100, thereby transporting the biological sample from the chamber 106 to the first section 101. The robotic arm 200 may put down the biological sample in the first section 101 of the laboratory 100. In some examples, the robotic arm 200 may place the biological sample on or within reach of the first conveyance platform 111. The robotic arm 200 may return to the chamber 106, such that the entire robotic arm 200 is within the chamber 106. The first door 107 of the chamber 106 may close, while the second door 108 remains closed. In some aspects, a portion of the robotic arm 200 that contacted the biological sample may be sanitized and/or sterilized at this point.

In some embodiments, the robotic arm 200 may put down and pick up the biological sample in the chamber 106, between transporting the biological sample to and from the chamber 106. In some embodiments, the robotic arm 200 may not put down and pick up the biological sample in the chamber 106, between transporting the biological sample to and from the chamber 106, but instead may hold the biological sample from when the biological sample is picked up in the second section 102 until the biological sample is put down in the first section 101.

In some embodiments, as FIG. 1 illustrates, the laboratory 100 may include more than one physical separation system 103. Thus, in some embodiments, the laboratory 100 may include multiple physical separation systems 103, each including the opening 104, the chamber 106 with the robotic arm 200 therein, the first door 107, and/or the second door 108.

In some embodiments, either the first section 101 or the second section 102 of the laboratory 100 may be at a higher air pressure that the other section. In some embodiments, when the first section 101 is a pre-amplification processing section and the second section 102 is a post-amplification processing section, the first section 101 of the laboratory 100 may be at a higher air pressure than the second section 102, such that any cross-contamination between sections of the laboratory 100 is cross-contamination of the second section 102 by airflow from the first section 101 to the second section 102.

FIG. 3 depicts a schematic top view of the portion of the laboratory of FIG. 1, with the physical separation system including two robotic arms, according to one or more embodiments of the present disclosure. As FIG. 3 illustrates, the physical separation system 103 may include a first robotic arm 201 in the first section 101 of the laboratory 100, and a second robotic arm 202 in the second section 102 of the laboratory 100. Although not shown, in other aspects, the first robotic arm 201 and the second robotic arm 202 may both be located within the chamber 106. In some embodiments, the first robotic arm 201 may transport the biological sample between the first section 101 and the chamber 106, and the second robotic arm 202 may transport the biological sample between the chamber 106 and the second section 102 of the laboratory 100. The first robotic arm 201 may pick up or place the biological sample from, on, or within reach of the first conveyance platform 111, and the second robotic arm 202 may pick up or place the biological sample from, on, or within reach of the second conveyance platform 112.

In some embodiments, to transport the biological sample from the first section 101 to the second section 102 of the laboratory 100, the first robotic arm 201 in the first section 101 may pick up the biological sample in the first section 101. In some examples, the first robotic arm 201 may pick up the biological sample from the first conveyance platform 111. The first door 107 of the chamber 106 may open while the second door 108 remains closed, such that the first section 101 of the laboratory 100 is isolated from the second section 102. At least a portion of the first robotic arm 201 may extend through the open first door 107 into the chamber 106, transporting the biological sample from the first section 101 of the laboratory 100 to the chamber 106. If the first robotic arm 201 is located within the chamber 106, then the first robotic arm 201 may extend through the first door 107 to pick up the biological sample in the first section 101 and then may bring the biological sample through the first door 107 and into the chamber 106. The first robotic arm 201 may put down the biological sample in the chamber 106. If the first robotic arm 201 is located in the first section 101, the first robotic arm 201 may return to the first section 101 of the laboratory 100, such that the first robotic arm 201 is fully within the first section 101. The first door 107 of the chamber 106 may close, while the second door 108 remains closed. In some embodiments, the exterior of the biological sample and/or one of the first robotic arm 201 or the second robotic arm 202 may be sanitized and/or sterilized within the chamber 106. The second door 108 of the chamber 106 may open, while the first door 107 remains closed. At least a portion of the second robotic arm 202 in the second section 102 of the laboratory 100 may extend through the open second door 108 into the chamber 106, and the second robotic arm 202 may pick up the biological sample from the chamber 106. If the second robotic arm 202 is located within the chamber 106, then the second robotic arm 202 may pick up the biological sample and then extend through the second door 108. The second robotic arm 202 may transport the biological sample from the chamber 106 to the second section 102 of the laboratory 100, and the second robotic arm 202 may put down the biological sample in the second section 102. In some examples, the second robotic arm 202 may place the biological sample on or within reach of the second conveyance platform 112. If located in the second section 102, the second robotic arm 202 may return to the second section 102 of the laboratory 100, such that the second robotic arm 202 is fully within the second section 102. The second door 108 of the chamber 106 may close, while the first door 107 remains closed. In some embodiments, the chamber 106, the first robotic arm 201, or the second robotic arm 202 may be sanitized and/or sterilized at any point during, before, and/or after transportation of the biological sample from the first section 101 to the second section 102 of the laboratory 100.

In some embodiments, to transport the biological sample from the second section 102 to the first section 101 of the laboratory 100, the second robotic arm 202 in the second section 102 may pick up the biological sample in the second section 102. In some examples, the second robotic arm 202 may pick up the biological sample from the second conveyance platform 112. The second door 108 of the chamber 106 may open while the first door 107 remains closed, such that the second section 102 of the laboratory 100 is isolated from the first section 101. At least a portion of the second robotic arm 202 holding the biological sample may extend through the open second door 108 into the chamber 106, transporting the biological sample from the second section 102 of the laboratory 100 to the chamber 106, and the second robotic arm 202 may put down the biological sample in the chamber 106. The second robotic arm 202 may return to the second section 102 of the laboratory 100, such that the second robotic arm 202 is fully within the second section 102. If the second robotic arm 202 is located within the chamber 106, then the second door 108 of the chamber 106 may open while the first door 107 remains closed, and the second robotic arm 202 may extend through the second door 108 to pick up the biological sample in the second section 102 and bring the biological sample through the second door 108 and into the chamber 106. The second door 108 of the chamber 106 may close, while the first door 107 remains closed. In some embodiments, the exterior of the biological sample and/or one of the first robotic arm 201 or the second robotic arm 202 may be sanitized and/or sterilized within the chamber 106. The first door 107 of the chamber 106 may open, while the second door 108 remains closed. At least a portion of the first robotic arm 201 in the first section 101 of the laboratory 100 may extend through the open first door 107 into the chamber 106, and the first robotic arm 201 may pick up the biological sample from the chamber 106. The first robotic arm 201 may transport the biological sample from the chamber 106 to the first section 101 of the laboratory 100, and the first robotic arm 201 put down the biological sample in the first section 101. In some examples, the first robotic arm 201 may place the biological sample on or within reach of the first conveyance platform 111. If the first robotic arm 201 is located within the chamber 106, then the first door 107 of the chamber 106 may open while the second door 108 remains closed, and the first robotic arm 201 may extend through the first door 107 to pick up the biological sample and bring the biological sample through the first door 107 and into the first section 101 of the laboratory 100. The first door 107 of the chamber 106 may close, while the second door 108 remains closed. In some embodiments, the chamber 106 and/or one of the first robotic arm 201 or the second robotic arm 202 may be sanitized and/or sterilized at any point during, before, and/or after transportation of the biological sample from the second section 102 to the first section 101 of the laboratory 100.

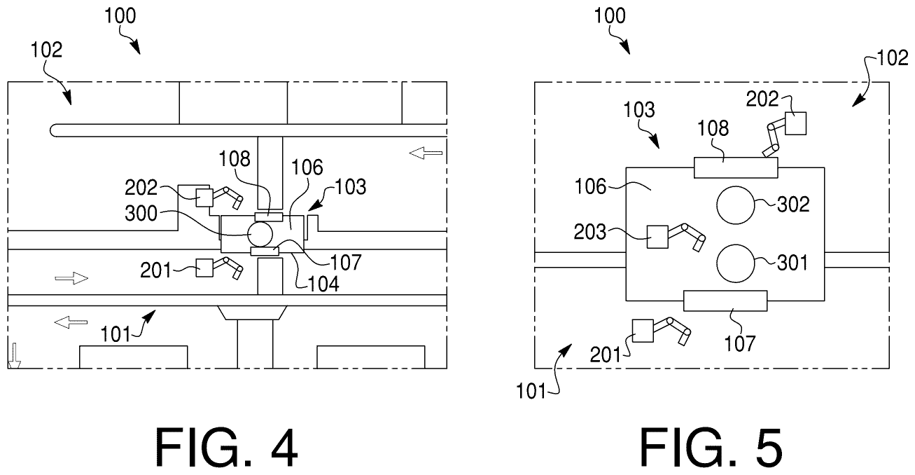

FIG. 4 depicts a schematic top view of the portion of the laboratory of FIG. 1, with the physical separation system including two robotic arms and a carousel, according to one or more embodiments of the present disclosure. As FIG. 4 illustrates, the physical separation system 103 may include the first robotic arm 201 in the first section 101 of the laboratory 100, the second robotic arm 202 in the second section 102 of the laboratory 100, and a carousel 300 in the chamber 106. Alternatively, as with FIG. 3, the first robotic arm 201 and the second robotic arm 202 may be located in the chamber 106, in addition to the carousel 300. In some embodiments, the first robotic arm 201 may transport the biological sample between the first section 101 of the laboratory 100 and the carousel 300, and the second robotic arm 202 may transport the biological sample between the carousel 300 and the second section 102 of the laboratory 100. The first robotic arm 201 may pick up or place the biological sample from, on, or within reach of the first conveyance platform 111, and the second robotic arm 202 may pick up or place the biological sample from, on, or within reach of the second conveyance platform 112. In some embodiments, the carousel 300 may include a plurality of openings (e.g., more than one opening), such as slots, holders, compartments, etc., for holding or otherwise for receiving biological samples. In some embodiments, the carousel 300 may be rotatable around an axis, such that the biological sample may be moved between a location in the chamber 106 proximate the first door 107 and a location in the chamber 106 proximate the second door 108.

In some embodiments, to transport the biological sample from the first section 101 to the second section 102 of the laboratory 100, the first robotic arm 201 in the first section 101 may pick up the biological sample in the first section 101. In some examples, the first robotic arm 201 may pick up the biological sample from the first conveyance platform 111. The first door 107 of the chamber 106 may open while the second door 108 remains closed, such that the first section 101 of the laboratory 100 is isolated from the second section 102. At least a portion of the first robotic arm 201 may extend through the open first door 107 into the chamber 106, transporting the biological sample from the first section 101 of the laboratory 100 into the chamber 106. If the first robotic arm 201 is located within the chamber 106, then the first door 107 of the chamber 106 may open while the second door 108 remains closed, and the first robotic arm 201 may extend through the first door 107 to pick up the biological sample and bring the biological sample through the first door 107 and into the chamber 106. The first robotic arm 201 may place the biological sample into an opening of the carousel 300 within the chamber 106. If located in the first section 101, the first robotic arm 201 may return to the first section 101 of the laboratory 100, such that the first robotic arm 201 is fully within the first section 101. The first door 107 of the chamber 106 may close, while the second door 108 remains closed. In some embodiments, the exterior of the biological sample and/or the first arm 201 or the second arm 202 may be sanitized and/or sterilized within the chamber 106. The carousel 300 may rotate so that the biological sample is moved from adjacent the first door 107 to adjacent the second door 108. The second door 108 of the chamber 106 may open, while the first door 107 remains closed. At least a portion of the second robotic arm 202 in the second section 102 of the laboratory 100 may extend through the open second door 108 into the chamber 106, and the second robotic arm 202 may pick up the biological sample from the carousel 300 in the chamber 106. If the second robotic arm 202 is located within the chamber 106, then the second door 108 of the chamber 106 may open while the first door 107 remains closed, and the second robotic arm 202 may pick up the biological sample from the carousel 300 and may extend through the second door 108. The second robotic arm 202 may transport the biological sample from the chamber 106 to the second section 102 of the laboratory 100, and put down the biological sample in the second section 102. In some examples, the second robotic arm 202 may place the biological sample on or within reach of the second conveyance platform 112. If located in the second section 102, the second robotic arm 202 may return to the second section 102 of the laboratory 100, such that the second robotic arm 202 is fully within the second section 102. If located within the chamber 106, the second robotic arm 202 may return to within the chamber 106. The second door 108 of the chamber 106 may close, while the first door 107 remains closed. In some embodiments, the chamber 106, the first robotic arm 201, the second robotic arm 202, and/or any component within the chamber 106 may be sanitized and/or sterilized at any point during, before, and/or after transportation of the biological sample from the first section 101 to the second section 102 of the laboratory 100.

In some embodiments, to transport the biological sample from the second section 102 to the first section 101 of the laboratory 100, the second robotic arm 202 in the second section 102 may pick up the biological sample in the second section 102. In some examples, the second robotic arm 202 may pick up the biological sample from the second conveyance platform 112. The second door 108 of the chamber 106 may open while the first door 107 remains closed, such that the second section 102 of the laboratory 100 is isolated from the first section 101. At least a portion of the second robotic arm 202 may extend through the open second door 108 into the chamber 106, transporting the biological sample from the second section 102 of the laboratory 100 to the chamber 106. If the second robotic arm 202 is located within the chamber 106, then the second door 108 of the chamber 106 may open while the first door 107 remains closed, and the second robotic arm 202 may extend through the second door 108 to pick up the biological sample and bring the biological sample through the second door 108 and into the chamber 106. The second robotic arm 202 may place the biological sample into an opening of the carousel 300 within the chamber 106. If located in the second section 102, the second robotic arm 202 may return to the second section 102 of the laboratory 100, such that the second robotic arm 202 is fully within the second section 102. If located within the chamber 106, the second robotic arm 202 may return to within the chamber 106. The second door 108 of the chamber 106 may close, while the first door 107 remains closed. In some embodiments, the exterior of the biological sample, the first robotic arm 201, and/or the second robotic arm 202 may be sanitized and/or sterilized within the chamber 106. The first door 107 of the chamber 106 may open, while the second door 108 remains closed. If located in the first section 101, at least a portion of the first robotic arm 201 in the first section 101 of the laboratory 100 may extend through the open first door 107 into the chamber 106, and the first robotic arm 201 may pick up the biological sample from the carousel 300 within the chamber 106. If the first robotic arm 201 is located within the chamber 106, then the first door 107 of the chamber 106 may open while the second door 108 remains closed, and the first robotic arm 201 may pick up the biological sample from the carousel 300 and may extend through the first door 107. The first robotic arm 201 may transport the biological sample from the chamber 106 to the first section 101 of the laboratory 100, and put down the biological sample in the first section 101. In some examples, the first robotic arm 201 may place the biological sample on or within reach of the first conveyance platform 111. If located in the first section 101, the first robotic arm 201 may return to the first section 101 of the laboratory 100, such that the first robotic arm 201 is fully within the first section 101. If the first robotic arm 201 is located within the chamber 106, the first robotic arm 201 may return to the chamber 106. The first door 107 of the chamber 106 may close, while the second door 108 remains closed. In some embodiments, the chamber 106, the first robotic arm 201, the second robotic arm 202, and/or any component within the chamber 106 may be sanitized and/or sterilized at any point during, before, and/or after transportation of the biological sample from the second section 102 to the first section 101 of the laboratory 100.

FIG. 5 depicts a schematic top view of the portion of the laboratory of FIG. 1, with the physical separation system including three robotic arms and two carousels, according to one or more embodiments of the present disclosure. As FIG. 5 illustrates, the physical separation system 103 may include the first robotic arm 201 in the first section 101 of the laboratory 100, the second robotic arm 202 in the second section 102 of the laboratory 100, and a third robotic arm 203 in the chamber 106. The physical separation system 103 may further include a first carousel 301 and a second carousel 302 in the chamber 106. In other aspects, the first robotic arm 201 and the second robotic arm 202 may also be located within the chamber 106, e.g., between the first door 107 and the first carousel 301, and between the second door 108 and the second carousel 302, respectively. In some embodiments, the first robotic arm 201 may transport the biological sample between the first section 101 of the laboratory 100 and the first carousel 301 within the chamber 106, the second robotic arm 202 may transport the biological sample between the second carousel 302 in the chamber 106 and the second section 102 of the laboratory 100, and the third robotic arm 203 may transport the biological sample between the first carousel 301 and the second carousel 302 within the chamber 106. The first robotic arm 201 may pick up or place the biological sample from, on, or within reach of the first conveyance platform 111 and the second robotic arm 202 may pick up or place the biological sample from, on, or within reach of the second conveyance platform 112.

In some embodiments, to transport the biological sample from the first section 101 to the second section 102 of the laboratory 100, the first robotic arm 201 in the first section 101 may pick up the biological sample in the first section 101. In some examples, the first robotic arm 201 may pick up the biological sample from the first conveyance platform 111. The first door 107 of the chamber 106 may open while the second door 108 remains closed, such that the first section 101 of the laboratory 100 is isolated from the second section 102. At least a portion of the first robotic arm 201 may extend through the open first door 107 into the chamber 106, transporting the biological sample from the first section 101 of the laboratory 100 to the chamber 106. If located within the chamber 106, the first robotic arm 201 may extend through the open first door 107, may pick up the biological sample in the first section 101, and may carry the biological sample into the chamber 106. The first robotic arm 201 may place the biological sample in an opening of the first carousel 301 within the chamber 106. If located in the first section 101, the first robotic arm 201 may return to the first section 101 of the laboratory 100, such that the first robotic arm 201 is fully within the first section 101. If located within the chamber 106, the first robotic arm 201 may return to within the chamber 106. The first door 107 of the chamber 106 may close, while the second door 108 remains closed. The first carousel 301 may rotate, thereby moving the biological sample from a location proximate the first door 107 of the chamber 106 to a location proximate the second carousel 302. The third robotic arm 203 may pick up the biological sample from the first carousel 301 within the chamber 106, and place the biological sample in an opening of the second carousel 302 within the chamber 106. The second carousel 302 may rotate, thereby moving the biological sample from the location proximate the first carousel 301 to a location proximate the second door 108 of the chamber 106. In some embodiments, the exterior of the biological sample, the first robotic arm 201, the second robotic arm 202, and/or the third robotic arm 203 may be sanitized and/or sterilized within the chamber 106. The second door 108 may open, while the first door 107 remains closed. If located in the second section 102, at least a portion of the second robotic arm 202 in the second section 102 of the laboratory 100 may extend through the open second door 108 into the chamber 106, and the second robotic arm 202 may pick up the biological sample from the second carousel 302 within the chamber 106. If located in the chamber 106, the second robotic arm 202 may pick up the biological sample from the second carousel 302 and extend through the second door 108 and into the second section 102. The second robotic arm 202 may transport the biological sample from the chamber 106 to the second section 102 of the laboratory 100, and put down the biological sample in the second section 102. In some examples, the second robotic arm 202 may place the biological sample on or within reach of the second conveyance platform 112. If the second robotic arm 202 is located in the second section 102, the second robotic arm 202 may return to the second section 102 of the laboratory 100, such that the second robotic arm 202 is fully within the second section 102. If the second robotic arm 202 is located within the chamber 106, then the second robotic arm may return to being fully within the chamber 106. The second door 108 of the chamber 106 may close, while the first door 107 remains closed. In some embodiments, any component may be sanitized and/or sterilized at any point during, before, and/or after transportation of the biological sample from the first section 101 to the second section 102 of the laboratory 100.

In some embodiments, to transport the biological sample from the second section 102 to the first section 101 of the laboratory 100, the second robotic arm 202 in the second section 102 of the laboratory 100 may pick up the biological sample in the second section 102. In some examples, the second robotic arm 202 may pick up the biological sample from the second conveyance platform 112. The second door 108 of the chamber 106 may open while the first door 107 remains closed, such that the second section 102 of the laboratory 100 is isolated from the first section 101. At least a portion of the second robotic arm 202 may extend through the open second door 108 into the chamber 106. If located within the chamber 106, the second robotic arm 202 may extend through the second door 108 and may pick up the biological sample in the second section 102. The second arm may transport the biological sample from the second section 102 of the laboratory 100 to the chamber 106, and the second robotic arm 202 may place the biological sample in an opening of the second carousel 302 within the chamber 106. If located in the second section 102, the second robotic arm 202 may return to the second section 102 of the laboratory 100, such that the second robotic arm 202 is fully within the second section 102. If located within the chamber 106, the second robotic arm 202 may return to within the chamber 106. The second door 108 of the chamber 106 may close, while the first door 107 remains closed. The second carousel 302 may rotate, thereby moving the biological sample from the location proximate the second door 108 of the chamber 106 to a location proximate the first carousel 301. The third robotic arm 203 may pick up the biological sample from the second carousel 302 within the chamber 106 and place the biological sample in the first carousel 301 within the chamber 106. The first carousel 301 may rotate, thereby moving the biological sample from the location proximate the second carousel 302 to a location proximate the first door 107 of the chamber 106. In some embodiments, the exterior of the biological sample, the first robotic arm 201, the second robotic arm 202, and/or the third robotic arm 203 may be sanitized and/or sterilized within the chamber 106. The first door 107 of the chamber 106 may open, while the second door 108 remains closed. At least a portion of the first robotic arm 201 in the first section 101 of the laboratory 100 may extend through the open first door 107 into the chamber 106, and the first robotic arm 201 may pick up the biological sample from the first carousel 301 in the chamber 106. If located in the chamber 106, the first robotic arm 201 may pick up the biological sample from the first carousel 301 in the chamber 106 and may extend through the open first door 107. The first robotic arm 201 may transport the biological sample from the chamber 106 to the first section 101 of the laboratory 100, and put down the biological sample in the first section 101. In some examples, the first robotic arm 201 may place the biological sample on or within reach of the first conveyance platform 111. If located in the first section 101, the first robotic arm 201 may return to the first section 101 of the laboratory 100, such that the first robotic arm 201 is fully within the first section 101. If located in the chamber 106, the first robotic arm 201 may return to being fully within the chamber 106. The first door 107 may close, while the second door 108 remains closed. In some embodiments, any component may be sanitized and/or sterilized at any point during, before, and/or after transportation of the biological sample from the second section 102 to the first section 101 of the laboratory 100.

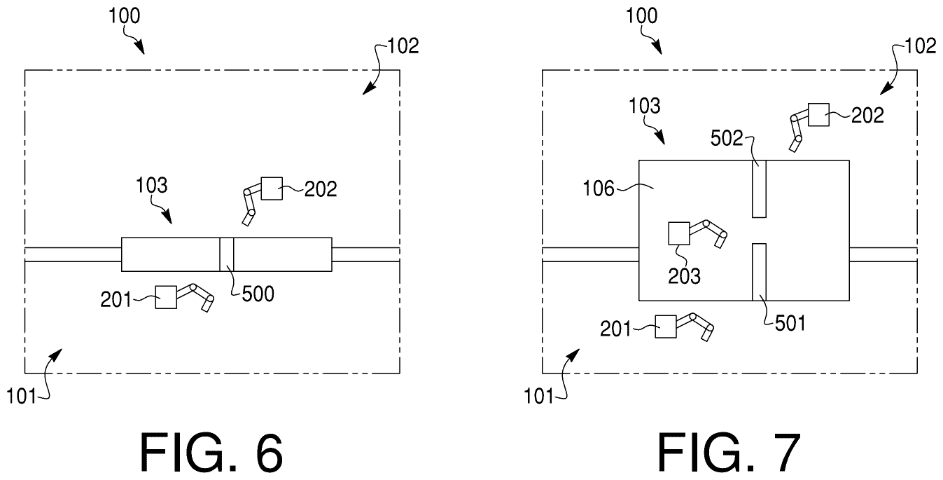

FIG. 6 depicts a schematic top view of the portion of the laboratory of FIG. 1, with the physical separation system including two robotic arms and a drawer, according to one or more embodiments of the present disclosure. As FIG. 6 illustrates, the physical separation system 103 may include the first robotic arm 201 in the first section 101 of the laboratory 100, the second robotic arm 202 in the second section 102 of the laboratory 100, and a drawer 500 between the first section 101 and the second section 102. In some embodiments, the first robotic arm 201 may transport the biological sample between the first section 101 of the laboratory 100 and the drawer 500, and the second robotic arm 202 may transport the biological sample between the drawer 500 and the second section 102 of the laboratory 100. The first robotic arm 201 may pick up or place the biological sample from, on, or within reach of the first conveyance platform 111, and the second robotic arm 202 may pick up or place the biological sample from, on, or within reach of the second conveyance platform 112.

In some embodiments, to transport the biological sample from the first section 101 to the second section 102 of the laboratory 100, the first robotic arm 201 in the first section 101 may pick up the biological sample in the first section 101. In some examples, the first robotic arm 201 may pick up the biological sample from the first conveyance platform 111. The drawer 500 may open such that an interior of the drawer 500 may be accessed from the first section 101 of the laboratory 100, while the drawer 500 remains closed to the second section 102 of the laboratory 100. The first robotic arm 201 may transport the biological sample from the first section 101 of the laboratory 100 to the drawer 500, and the first robotic arm 201 may place the biological sample in the drawer 500. The first robotic arm 201 may withdraw from the drawer 500, such that no portion of the first robotic arm 201 is within the drawer 500. The drawer 500 may close, thereby isolating the first section 101 of the laboratory 100 from the drawer 500 and the second section 102. In some embodiments, the exterior of the biological sample may be sanitized and/or sterilized within the drawer 500. The drawer 500 may open such that the interior of the drawer 500 may be accessed from the second section 102 of the laboratory 100, while the drawer 500 remains closed to the first section 101 of the laboratory 100. The second robotic arm 202 may pick up the biological sample from the drawer 500. The second robotic arm 202 may withdraw from the drawer 500, such that no portion of the second robotic arm 202 is within the drawer 500. The drawer 500 may close, thereby isolating the first section 101 of the laboratory 100 from the drawer 500 and the second section 102. The second robotic arm 202 may put down the biological sample in the second section 102 of the laboratory, thereby transporting the biological sample from the drawer 500 to the second section 102. In some examples, the second robotic arm 202 may place the biological sample on or within reach of the second conveyance platform 112. In some embodiments, any component may be sanitized and/or sterilized at any point during, before, and/or after transportation of the biological sample from the first section 101 to the second section 102 of the laboratory 100.

In some embodiments, to transport the biological sample from the second section 102 to the first section 101 of the laboratory 100, the second robotic arm 202 in the second section 102 may pick up the biological sample in the second section 102. In some examples, the second robotic arm 202 may pick up the biological sample from the second conveyance platform 112. The drawer 500 may open such that an interior of the drawer 500 may be accessed from the second section 102 of the laboratory 100, while remaining closed to the first section 101. The second robotic arm 202 may transport the biological sample from the second section 102 of the laboratory 100 to the drawer 500, and the second robotic arm 202 may place the biological sample in the drawer 500. The second robotic arm 202 may withdraw from the drawer 500, such that no portion of the second robotic arm 202 is within the drawer 500. The drawer 500 may close, thereby isolating the second section 102 of the laboratory 100 from the drawer 500 and the first section 101. In some embodiments, the exterior of the biological sample may be sanitized and/or sterilized within the drawer 500. The drawer 500 may open such that the interior of the drawer 500 may be accessed from the first section 101 of the laboratory 100. The first robotic arm 201 may pick up the biological sample from the drawer 500. The first robotic arm 201 may withdraw from the drawer 500, such that no portion of the first robotic arm 201 is within the drawer 500. The drawer 500 may close, thereby isolating the second section 102 of the laboratory 100 from the drawer 500 and the first section 101. The first robotic arm 201 may put down the biological sample in the first section 101 of the laboratory 100, thereby transporting the biological sample from the drawer 500 to the first section 101. In some examples, the first robotic arm 201 may place the biological sample on or within reach of the first conveyance platform 111. In some embodiments, any component may be sanitized and/or sterilized at any point during, before, and/or after transportation of the biological sample from the second section 102 to the first section 101 of the laboratory 100.

FIG. 7 depicts a schematic top view of the portion of the laboratory of FIG. 1, with the physical separation system including three robotic arms and two drawers, according to one or more embodiments of the present disclosure. As FIG. 7 illustrates, the physical separation system 103 may include the first robotic arm 201 in the first section 101 of the laboratory 100, the second robotic arm 202 in the second section 102 of the laboratory 100, and a third robotic arm 203 in the chamber 106. The physical separation system 103 may further include a first drawer 501 and a second drawer 502, each of which is accessible from within the chamber 106. In some embodiments, the first robotic arm 201 may transport the biological sample between the first section 101 of the laboratory 100 and the first drawer 501, the second robotic arm 202 may transport the biological sample between the second drawer 502 and the second section 102 of the laboratory 100, and the third robotic arm 203 may transport the biological sample between the first drawer 501 and the second drawer 502 from within the chamber 106. The first robotic arm 201 may pick up or place the biological sample from, on, or within reach of the first conveyance platform 111, and the second robotic arm 202 may pick up or place the biological sample from, on, or within reach of the second conveyance platform 112.

In some embodiments, to transport the biological sample from the first section 101 to the second section 102 of the laboratory 100, the first robotic arm 201 in the first section 101 may pick up the biological sample in the first section 101. In some examples, the first robotic arm 201 may pick up the biological sample from the first conveyance platform 111. The first drawer 501 may open such that an interior of the first drawer 501 may be accessed from the first section 101 while remaining closed to the chamber 106, and while the second drawer 502 remains closed. The first robotic arm 201 may transport the biological sample from the first section 101 of the laboratory 100 to the first drawer 501, and the first robotic arm 201 may place the biological sample in the first drawer 501. The first robotic arm 201 may withdraw from the first drawer 501, such that no portion of the first robotic arm 201 is within the first drawer 501. The first drawer 501 may close, thereby isolating the first section 101 of the laboratory 100 from the chamber 106 and the second section 102. In some embodiments, the exterior of the biological sample may be sanitized and/or sterilized within the first drawer 501. The first drawer 501 and the second drawer 502 may open so as to be accessible to the chamber 106, while remaining closed to the first section 101 and the second section 102 of the laboratory 100. The third robotic arm 203 may pick up the biological sample from the interior of the first drawer 501 within the chamber 106, and place the biological sample within an interior of the second drawer 502 within the chamber 106. In other aspects, only the first drawer 501 may open so as to be accessible to the chamber 106, while remaining closed to the first section 101, while the second drawer 502 may remain closed. The third robotic arm 203 may pick up the biological sample from the interior of the first drawer 501 within the chamber 106, and the first drawer 501 may close. Once the first drawer 501 is closed, the second drawer 502 may open so as to be accessible to the chamber 106, and the third robotic arm 203 may place the biological sample into the second drawer 502 in the chamber 106. In some embodiments, the exterior of the biological sample may be sanitized and/or sterilized within chamber 106 and/or within the second drawer 502. The first drawer 501 and the second drawer 502 may close so as to be inaccessible from the chamber 106, and the first section 101 and the second section 102 of the laboratory 100. The second drawer 502 may open such that the interior of the second drawer 502 may be accessed from the second section 102 of the laboratory 100 while remaining closed to the chamber 106, and while the first drawer 501 remains closed. The second robotic arm 202 may pick up the biological sample from the interior of the second drawer 502 and place the biological sample in the second section 102 of the laboratory 100, thereby transporting the biological sample from the second drawer 502 to the second section 102. In some examples, the second robotic arm 202 may place the biological sample on or within reach of the second conveyance platform 112. The second robotic arm 202 may withdraw from the second drawer 502, such that no portion of the second robotic arm 202 is within the second drawer 502. The second drawer 502 may close, thereby isolating the second drawer 502 from the chamber 106, the first section 101, and the second section 102 of the laboratory 100. In some embodiments, any component may be sanitized and/or sterilized at any point during, before, and/or after transportation of the biological sample from the first section 101 to the second section 102 of the laboratory 100.

In some embodiments, to transport the biological sample from the second section 102 to the first section 101 of the laboratory 100, the second robotic arm 202 in the second section 102 may pick up the biological sample in the second section 102. In some examples, the second robotic arm 202 may pick up the biological sample from the second conveyance platform 112. The second drawer 502 may open such that the interior of the second drawer 502 may be accessed from the second section 102 while remaining closed to the chamber 106, and while the first drawer 501 remains closed. The second robotic arm 202 may transport the biological sample from the second section 102 of the laboratory 100 to the second drawer 502, and the second robotic arm 202 may place the biological sample in the second drawer 502. The second robotic arm 202 may withdraw from the second drawer 502, such that no portion of the second robotic arm 202 is within the second drawer 502. The second drawer 502 may close, thereby isolating the second section 102 of the laboratory 100 from the chamber 106 and the first section 101. In some embodiments, the exterior of the biological sample may be sanitized and/or sterilized within the second drawer 502. The first drawer 501 and the second drawer 502 may open so as to be accessible to the chamber 106, while remaining closed to the first section 101 and the second section 102 of the laboratory 100. The third robotic arm 203 may pick up the biological sample from the interior of the second drawer 502 within the chamber 106, and place the biological sample within the interior of the first drawer 501 within the chamber 106. The first drawer 501 and the second drawer 502 may close so as to be inaccessible from the chamber 106, and the first section 101 and the second section 102 of the laboratory 100. In other aspects, only the second drawer 502 may open so as to be accessible to the chamber 106, while remaining closed to the second section 102, while the first drawer 501 may remain closed. The third robotic arm 203 may pick up the biological sample from the interior of the second drawer 502 within the chamber 106, and the second drawer 502 may close. Once the second drawer 502 is closed, the first drawer 501 may open so as to be accessible to the chamber 106, and the third robotic arm 203 may place the biological sample into the first drawer 501 in the chamber 106. In some embodiments, the exterior of the biological sample may be sanitized and/or sterilized within the chamber 106 and/or within the first drawer 501. The first drawer 501 may open such that the interior of the first drawer 501 may be accessed from the first section 101 while remaining closed to the chamber 106, and while the second drawer 502 remains closed. The first robotic arm 201 may pick up the biological sample from the interior of the first drawer 501 and place the biological sample in the first section 101 of the laboratory 100, thereby transporting the biological sample from the first drawer 501 to the first section 101. In some examples, the first robotic arm 201 may place the biological sample on or within reach of the first conveyance platform 111. The first robotic arm 201 may withdraw from the first drawer 501, such that no portion of the first robotic arm 201 is within the first drawer 501. The first drawer 501 may close, thereby isolating the first drawer 501 from the chamber 106, the first section 101, and the second section 102 of the laboratory 100. In some embodiments, any component may be sanitized and/or sterilized at any point during, before, and/or after transportation of the biological sample from the second section 102 to the first section 101 of the laboratory 100.

In some embodiments, when the first drawer 501 is accessible from the first section 101 of the laboratory 100, the structure of the first drawer 501 itself, and/or a door mechanism, may prevent access to the interior of the first drawer 501 from inside the chamber 106. In some embodiments, when the first drawer 501 is accessible from the first section 101 of the laboratory 100, the interior of the first drawer 501 may still be accessed from inside the chamber 106. In some embodiments, when the second drawer 502 is accessible from the second section 102 of the laboratory 100, the structure of the second drawer 502 itself, and/or a door mechanism, may prevent access to the interior of the second drawer 502 from inside the chamber 106. In some embodiments, when the second drawer 502 is accessible from the second section 102 of the laboratory 100, the interior of the second drawer 502 may still be accessed from inside the chamber 106.

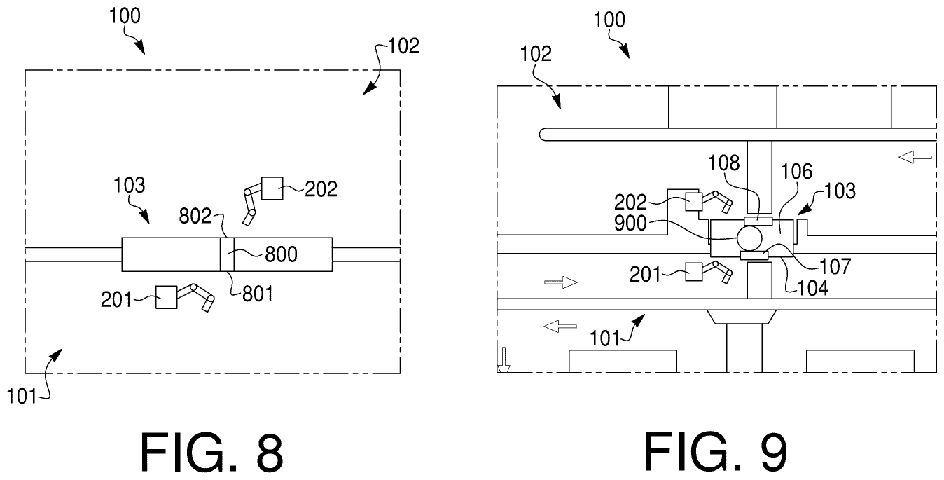

FIG. 8 depicts a schematic top view of the portion of the laboratory of FIG. 1, with the physical separation system including two robotic arms and a pneumatic tube, according to one or more embodiments of the present disclosure. As FIG. 8 illustrates, the physical separation system 103 may include the first robotic arm 201 in the first section 101 of the laboratory 100, the second robotic arm 202 in the second section 102 of the laboratory 100, and a pneumatic tube 800 between the first section 101 and the second section 102. In some embodiments, the first robotic arm 201 may transport the biological sample between the first section 101 of the laboratory 100 and the pneumatic tube 800, the second robotic arm 202 may transport the biological sample between the pneumatic tube 800 and the second section 102 of the laboratory 100, and the pneumatic tube 800 may transport the biological sample between the first section 101 and the second section 102 of the laboratory. The first robotic arm 201 may pick up or place the biological sample from, on, or within reach of the first conveyance platform 111 and the second robotic arm 202 may pick up or place the biological sample from, on, or within reach of the second conveyance platform 112. In some embodiments, the pneumatic tube 800 may or may not be within the chamber 106. For example, the pneumatic tube 800 may connect the first section 101 and the second section 102 when they are not physically adjacent to one another, such as when the first section 101 and the section 102 are in different rooms, on different floors of the same building, or in different buildings.

As FIG. 8 illustrates, the pneumatic tube 800 may include a first door 801, which may open and close to permit and prevent communication or exposure between the pneumatic tube 800 and the first section 101. The pneumatic tube 800 may include a second door 802, which may open and close to permit and prevent communication or exposure between the pneumatic tube 800 and the second section 102.

In some embodiments, to transport the biological sample from the first section 101 to the second section 102 of the laboratory 100, the first robotic arm 201 in the first section 101 may pick up the biological sample in the first section 101, and place the biological sample in a pneumatic carrier. Alternatively, in some embodiments, the biological sample may be placed inside the pneumatic carrier by a person or mechanism other than the first robotic arm 201. The first door 801 of the pneumatic tube 800 may open, while the second door 802 of the pneumatic tube 800 remains closed, thereby isolating the first section 101 of the laboratory 100 from the second section 102. The first robotic arm 201 may pick up the pneumatic carrier including the biological sample, and place the pneumatic carrier in the pneumatic tube 800. In some examples, the first robotic arm 201 may pick up the pneumatic tube and/or biological sample from the first conveyance platform 111. The first robotic arm 201 may withdraw from the pneumatic tube 800, such that no portion of the first robotic arm 201 is within the pneumatic tube 800. The first door 801 may close, thereby isolating the first section 101 of the laboratory 100 from the second section 102. The pneumatic tube 800 may transport the pneumatic carrier including the biological sample from a location proximate the first section 101 to a location proximate the second section 102 of the laboratory 100. The second door 802 of the pneumatic tube 800 may open, while the first door 801 of the pneumatic tube 800 remains closed. The second robotic arm 202 may pick up the pneumatic carrier including the biological sample from the pneumatic tube 800. The second robotic arm 202 may put down the pneumatic carrier including the biological sample in the second section 102 of the laboratory 100, thereby transporting the biological sample from pneumatic tube 800 to the second section 102. In some examples, the second robotic arm 202 may place the pneumatic carrier and/or biological sample on or within reach of the second conveyance platform 112. The second robotic arm 202 may withdraw from the pneumatic tube 800, such that no portion of the second robotic arm 202 is within the pneumatic tube 800. The second door 802 may close, thereby isolating the first section 101 of the laboratory 100 from the second section 102. The second robotic arm 202, another mechanism, or a person, may remove the biological sample from the pneumatic carrier. In some embodiments, the pneumatic carrier including the biological sample may be sanitized or sterilized within the pneumatic tube 800. In some embodiments, the pneumatic tube 800, the first robotic arm 201, the second robotic arm 202, and/or any component within the chamber 106 may be sanitized and/or sterilized at any point during, before, and/or after transportation of the biological sample from the first section 101 to the second section 102 of the laboratory 100. In some embodiments, after removal of the biological sample from the pneumatic carrier, the pneumatic carrier is sanitized and/or sterilized prior to reuse.

In some embodiments, to transport the biological sample from the second section 102 to the first section 101 of the laboratory 100, the second robotic arm 202 in the second section 102 may pick up the biological sample in the second section 102, and place the biological sample in the pneumatic carrier. Alternatively, in some embodiments, the biological sample may be placed inside the pneumatic carrier by a person or mechanism other than the second robotic arm 202. The second door 802 of the pneumatic tube 800 may open, while the first door 801 of the pneumatic tube 800 remains closed, thereby isolating the second section 102 of the laboratory 100 from the first section 101. The second robotic arm 202 may pick up the pneumatic carrier including the biological sample, and place the pneumatic carrier in the pneumatic tube 800. In some examples, the second robotic arm 202 may pick up the pneumatic tube and/or biological sample from the second conveyance platform 112. The second robotic arm 202 may withdraw from the pneumatic tube 800, such that no portion of the second robotic arm 202 is within the pneumatic tube 800. The second door 802 may close, thereby isolating the second section 102 of the laboratory 100 from the first section 101. The pneumatic tube 800 may transport the pneumatic carrier including the biological sample from the location proximate the second section 102 to the location proximate the first section 101 of the laboratory 100. The first door 801 of the pneumatic tube 800 may open, while the second door 802 of the pneumatic tube 800 remains closed. The first robotic arm 201 may pick up the pneumatic carrier including the biological sample from the pneumatic tube 800 and transport the biological sample from pneumatic tube 800 to the first section 101 of the laboratory 100, and put down the pneumatic carrier including the biological sample in the first section 101. In some examples, the first robotic arm 201 may place the pneumatic carrier and/or biological sample on or within reach of the first conveyance platform 111. The first robotic arm 201 may withdraw from the pneumatic tube 800, such that no portion of the first robotic arm 201 is within the pneumatic tube 800. The first door 801 may close, thereby isolating the second section 102 of the laboratory 100 from the first section 101. The first robotic arm 201, another mechanism, or a person may remove the biological sample from the pneumatic carrier. In some embodiments, the pneumatic carrier including the biological sample may be sanitized or sterilized within the pneumatic tube 800. In some embodiments, the physical separation system 103 and/or any component of the physical separation system 103 may be sanitized and/or sterilized at any point during, before, and/or after transportation of the biological sample from the second section 102 to the first section 101 of the laboratory 100. In some embodiments, after removal of the biological sample from the pneumatic carrier, the pneumatic carrier is sanitized and/or sterilized prior to reuse.

FIG. 9 depicts a schematic top view of the portion of the laboratory of FIG. 1, with the physical separation system including two robotic arms and a turntable, according to one or more embodiments of the present disclosure. As FIG. 9 illustrates, the physical separation system 103 may include the first robotic arm 201 in the first section 101 of the laboratory 100, the second robotic arm 202 in the second section 102 of the laboratory 100, and a turntable 900 within the chamber 106. In other aspects, the first robotic arm 201 and the second robotic arm 202 may also be located within the chamber 106, with the first robotic arm 201 being positioned between the first door 107 and the turntable 900, and the second robotic arm 202 being positioned between the second door 108 and the turntable 900. In some embodiments, the first robotic arm 201 may transport the biological sample between the first section 101 of the laboratory 100 and the turntable 900, and the second robotic arm 202 may transport the biological sample between the turntable 900 and the second section 102 of the laboratory 100.

The first robotic arm 201 may pick up or place the biological sample from, on, or within reach of the first conveyance platform 111, and the second robotic arm 202 may pick up or place the biological sample from, on, or within reach of the second conveyance platform 112. In some embodiments, the turntable 900 may include a surface, which may be a substantially flat top surface on which the biological sample is placed and/or from which the biological sample is picked up. In some embodiments, the turntable 900 may be rotatable about an axis. In some aspects, the top surface of the turntable may have an indent onto which the sample is placed.

In some embodiments, to transport the biological sample from the first section 101 to the second section 102 of the laboratory 100, the first robotic arm 201 in the first section 101 may pick up the biological sample in the first section 101. In some examples, the first robotic arm 201 may pick up the biological sample from the first conveyance platform 111. The first door 107 of the chamber 106 may open while the second door 108 remains closed, such that the first section 101 of the laboratory 100 is isolated from the second section 102. At least a portion of the first robotic arm 201 may extend through the open first door 107 into the chamber 106, transporting the biological sample from the first section 101 into the chamber 106, and the first robotic arm 201 may place the biological sample onto the surface of the turntable 900 within the chamber 106. The first robotic arm 201 may return to the first section 101 of the laboratory 100, such that the first robotic arm 201 is fully within the first section 101. If the first robotic arm 201 is located within the chamber 106, the first door 107 may open, and the first robotic arm 201 may extend through the open first door 107, may pick up the sample in the first section 101, and may withdraw through the first door 107 and back into the chamber 106, where the first robotic arm 201 may place the biological sample onto the surface of the turntable 900. The first door 107 of the chamber 106 may close, while the second door 108 remains closed. In some embodiments, the exterior of the biological sample may be sanitized and/or sterilized within the chamber 106. The turntable 900 within the chamber 106 may rotate to transport the biological sample from a location proximate the first section 101 to a location proximate the second section 102 of the laboratory 100. The second door 108 of the chamber 106 may open, while the first door 107 remains closed. If the second robotic arm 202 is located in the second section 102, at least a portion of the second robotic arm 202 in the second section 102 of the laboratory 100 may extend through the open second door 108 into the chamber 106, and the second robotic arm 202 may pick up the biological sample from the surface of the turntable 900 within the chamber 106. The second robotic arm 202 may put down the biological sample in the second section 102 of the laboratory 100, thereby transporting the biological sample from the chamber 106 to the second section 102. The second robotic arm 202 may return to the second section 102 of the laboratory 100, such that the second robotic arm 202 is fully within the second section 102. If the second robotic arm 202 is located within the chamber 106, the second robotic arm 202 may pick up the biological sample from the turntable 900, the second door 108 may open, and the second robotic arm 202 may extend through the open second door 108 and place the biological sample in the second section 102 of the laboratory 100. The second robotic arm 202 may then withdraw through the second door 108 back into the chamber 106. In some examples, the second robotic arm 202 may place the biological sample on or within reach of the second conveyance platform 112. The second door 108 of the chamber 106 may close, while the first door 107 remains closed. In some embodiments, the chamber 106 and/or any component within the chamber 106 may be sanitized and/or sterilized at any point during, before, and/or after transportation of the biological sample from the first section 101 to the second section 102 of the laboratory 100.

In some embodiments, to transport the biological sample from the second section 102 to the first section 101 of the laboratory 100, the second robotic arm 202 in the second section 102 may pick up the biological sample in the second section 102. In some examples, the second robotic arm 202 may pick up the biological sample from the second conveyance platform 112. The second door 108 of the chamber 106 may open while the first door 107 remains closed, such that the second section 102 of the laboratory 100 is isolated from the first section 101. At least a portion of the second robotic arm 202 may extend through the open second door 108 into the chamber 106, transporting the biological sample from the second section 102 to the chamber 106, and the second robotic arm 202 may place the biological sample onto the surface of the turntable 900 within the chamber 106. The second robotic arm 202 may return to the second section 102 of the laboratory 100, such that the second robotic arm 202 is fully within the second section 102. If the second robotic arm 202 is located within the chamber 106, the second door 108 may open, and the second arm 202 may extend through the open second door 108, may pick up the sample in the second section 102, and may withdraw through the second door 108 and back into the chamber 106, where the second robotic arm 202 may place the biological sample onto the surface of the turntable 900. The second door 108 of the chamber 106 may close, while the first door 107 remains closed. In some embodiments, the exterior of the biological sample may be sanitized and/or sterilized within the chamber 106. The turntable 900 within the chamber 106 may rotate to transport the biological sample from the location proximate the second section 102 of the laboratory 100 to the location proximate the first section 101. The first door 107 may open, while the second door 108 remains closed. At least a portion of the first robotic arm 201 in the first section 101 may extend through the open first door 107 into the chamber 106, and the first robotic arm 201 may pick up the biological sample from the surface of the turntable 900 within the chamber 106. The first robotic arm 201 may transport the biological sample from the chamber 106 to the first section 101, and put down the biological sample in the first section 101. The first robotic arm 201 may return to the first section 101 of the laboratory 100, such that the first robotic arm 201 is fully within the first section 101. If the first robotic arm 201 is located within the chamber 106, the first robotic arm 201 may pick up the biological sample from the turntable 900, the first door 107 may open, and the first robotic arm 201 may extend through the open first door 107 and place the biological sample in the first section 101 of the laboratory 100. The first robotic arm 201 may then withdraw through the first door 107 back into the chamber 106. In some examples, the first robotic arm 201 may place the biological sample on or within reach of the first conveyance platform 111. The first door 107 may close, while the second door 108 remains closed. In some embodiments, the chamber 106 and/or any component within the chamber 106 may be sanitized and/or sterilized at any point during, before, and/or after transportation of the biological sample from the second section 102 to the first section 101 of the laboratory 100.

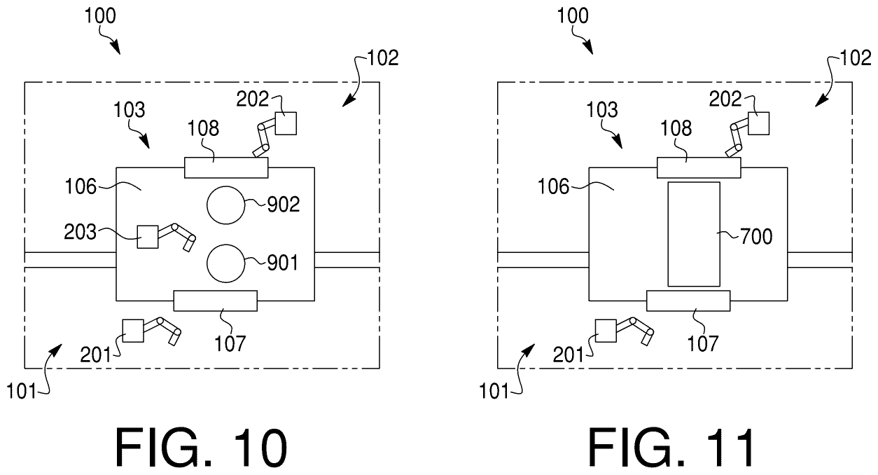

FIG. 10 depicts a schematic top view of the portion of the laboratory of FIG. 1, with the physical separation system including three robotic arms and two turntables, according to one or more embodiments of the present disclosure. As FIG. 10 illustrates, the physical separation system 103 may include the first robotic arm 201 in the first section 101 of the laboratory 100, the second robotic arm 202 in the second section 102 of the laboratory 100, and a third robotic arm 203 in the chamber 106. The physical separation system 103 may further include a first turntable 901 and a second turntable 902 within the chamber 106. In some aspects, the first robotic arm 201 and the second robotic arm 202 may also be located in the chamber 106 with the first turntable 901 and the second turntable 902. In some embodiments, the first robotic arm 201 may transport the biological sample between the first section 101 of the laboratory 100 and the first turntable 901, the second robotic arm 202 may transport the biological sample between the second turntable 902 and the second section 102 of the laboratory 100, and the third robotic arm 203 may transport the biological sample between the first turntable 901 and the second turntable 902 within the chamber 106. The first robotic arm 201 may pick up or place the biological sample from, on, or within reach of the first conveyance platform 111, and the second robotic arm 202 may pick up or place the biological sample from, on, or within reach of the second conveyance platform 112.

In some embodiments, to transport the biological sample from the first section 101 to the second section 102 of the laboratory 100, the first robotic arm 201 in the first section 101 may pick up the biological sample in the first section 101. In some examples, the first robotic arm 201 may pick up the biological sample from the first conveyance platform 111. The first door 107 of the chamber 106 may open while the second door 108 remains closed, such that the first section 101 of the laboratory 100 is isolated from the second section 102. At least a portion of the first robotic arm 201 may extend through the open first door 107 into the chamber 106, transporting the biological sample from the first section 101 to the chamber 106. The first robotic arm 201 may place the biological sample onto the first turntable 901 within the chamber 106. The first robotic arm 201 may return to the first section 101 of the laboratory 100, such that the first robotic arm 201 is fully within the first section 101. If the first robotic arm 201 is located within the chamber 106, the first door 107 may open, and the first robotic arm 201 may extend through the open first door 107, may pick up the sample in the first section 101, and may withdraw through the first door 107 and back into the chamber 106, where the first robotic arm 201 may place the biological sample onto the surface of the first turntable 901. The first door 107 may close, while the second door 108 remains closed. In some embodiments, the exterior of the biological sample, the first robotic arm 201, and/or the second robotic arm 202 may be sanitized and/or sterilized within the chamber 106. The first turntable 901 within the chamber 106 may rotate to transport the biological sample from a location proximate the first section 101 to a location proximate the second turntable 902. The third robotic arm 203 may pick up the biological sample from the first turntable 901 within the chamber 106, and place the biological sample onto the second turntable 902 within the chamber 106. The second turntable 902 within the chamber 106 may rotate to transport the biological sample from the location proximate the first turntable 901 to a location proximate the second section 102 of the laboratory 100. The second door 108 may open, while the first door 107 remains closed. At least a portion of the second robotic arm 202 in the second section 102 may extend through the open second door 108 into the chamber 106, and the second robotic arm 202 may pick up the biological sample from the second turntable 902 within the chamber 106. The second robotic arm 202 may transport the biological sample from the chamber 106 to the second section 102, and put down the biological sample in the second section 102. The second robotic arm 202 may return to the second section 102 of the laboratory 100, such that the second robotic arm 202 is fully within the second section 102. If the second robotic arm 202 is located within the chamber 106, the second robotic arm 202 may pick up the biological sample from the second turntable 902, the second door 108 may open, and the second robotic arm 202 may extend through the open second door 108 and place the biological sample in the second section 102 of the laboratory 100. The second robotic arm 202 may then withdraw through the second door 108 back into the chamber 106. In some examples, the second robotic arm 202 may place the biological sample on or within reach of the second conveyance platform 112. The second door 108 may close, while the first door 107 remains closed. In some embodiments, the chamber 106 and/or any component within the chamber 106 may be sanitized and/or sterilized at any point during, before, and/or after transportation of the biological sample from the first section 101 to the second section 102 of the laboratory 100.

In some embodiments, to transport the biological sample from the second section 102 to the first section 101 of the laboratory 100, the second robotic arm 202 in the second section 102 may pick up the biological sample in the second section 102. In some examples, the second robotic arm 202 may pick up the biological sample from the second conveyance platform 112. The second door 108 of the chamber 106 may open while the first door 107 remains closed, such that the second section 102 of the laboratory 100 is isolated from the first section 101. At least a portion of the second robotic arm 202 may extend through the open second door 108 into the chamber 106, transporting the biological sample from the second section 102 to the chamber 106, and the second robotic arm 202 may place the biological sample onto the second turntable 902 within the chamber 106. The second robotic arm 202 may return to the second section 102 of the laboratory 100, such that the second robotic arm 202 is fully within the second section 102. If the second robotic arm 202 is located within the chamber 106, the second door 108 may open, and the second robotic arm 202 may extend through the open second door 108, may pick up the sample in the second section 102, and may withdraw through the second door 108 back into the chamber 106, where the second robotic arm 202 may place the biological sample onto the surface of the second turntable 902.

The second door 108 may close, while the first door 107 remains closed. In some embodiments, the exterior of the biological sample, the first robotic arm 201, and/or the second robotic arm 202 may be sanitized and/or sterilized within the chamber 106. The second turntable 902 within the chamber 106 may rotate to transport the biological sample from the location proximate the second section 102 to the location proximate the first turntable 901. The third robotic arm 203 may pick up the biological sample from the second turntable 902 within the chamber 106, and place the biological sample onto the first turntable 901 within the chamber 106. The first turntable 901 within the chamber 106 may rotate to transport the biological sample from the location proximate the second turntable 902 to the location proximate the first section 101 of the laboratory 100. The first door 107 may open, while the second door 108 remains closed. At least a portion of the first robotic arm 201 in the first section 101 may extend through the open first door 107 into the chamber 106, and the first robotic arm 201 may pick up the biological sample from the first turntable 901 within the chamber 106. The first robotic arm 201 may transport the biological sample from the chamber 106 to the first section 101, and put down the biological sample in the first section 101. The first robotic arm 201 may return to the first section 101 of the laboratory 100, such that the first robotic arm 201 is fully within the first section 101. If the first robotic arm 201 is located within the chamber 106, the first robotic arm 201 may pick up the biological sample from the first turntable 901, the first door 107 may open, and the first robotic arm 201 may extend through the open first door 107 and place the biological sample in the first section 101 of the laboratory 100. The first robotic arm 201 may then withdraw through the first door 107 back into the chamber 106. In some examples, the first robotic arm 201 may place the biological sample on or within reach of the first conveyance platform 111. The first door 107 may close, while the second door 108 remains closed. In some embodiments, the chamber 106 and/or any component within the chamber 106 may be sanitized and/or sterilized at any point during, before, and/or after transportation of the biological sample from the second section 102 to the first section 101 of the laboratory 100.

FIG. 11 depicts a schematic top view of the portion of the laboratory of FIG. 1, with the physical separation system including two robotic arms and a conveyor, according to one or more embodiments of the present disclosure. As FIG. 11 illustrates, the physical separation system 103 may include the first robotic arm 201 in the first section 101 of the laboratory 100, the second robotic arm 202 in the second section 102 of the laboratory 100, and a conveyor 700 within the chamber 106. In other aspects, the first robotic arm 201 and the second robotic arm 202 may be located within the chamber 106 with the conveyor 700. In some embodiments, the first robotic arm 201 may transport the biological sample between the first section 101 of the laboratory 100 and a surface of the conveyor 700, and the second robotic arm 202 may transport the biological sample between the surface of the conveyor 700 and the second section 102 of the laboratory 100. The first robotic arm 201 may pick up or place the biological sample from, on, or within reach of the first conveyance platform 111, and the second robotic arm 202 may pick up or place the biological sample from, on, or within reach of the second conveyance platform 112.

In some embodiments, to transport the biological sample from the first section 101 to the second section 102 of the laboratory 100, the first robotic arm 201 in the first section 101 may pick up the biological sample in the first section 101. In some examples, the first robotic arm 201 may pick up the biological sample from the first conveyance platform 111. The first door 107 of the chamber 106 may open while the second door 108 remains closed, such that the first section 101 of the laboratory 100 is isolated from the second section 102. At least a portion of the first robotic arm 201 may extend through the open first door 107 into the chamber 106, transporting the biological sample from the first section 101 into the chamber 106, and the first robotic arm 201 may place the biological sample onto the surface of the conveyor 700 within the chamber 106. The first robotic arm 201 may return to the first section 101 of the laboratory 100, such that the first robotic arm 201 is fully within the first section 101. If the first robotic arm 201 is located within the chamber 106, the first door 107 may open, the first robotic arm 201 may extend through the open first door 107, and the first robotic arm 201 may pick up the biological sample in the first section 101 of the laboratory 100. The first robotic arm 201 may then withdraw through the first door 107 back into the chamber 106 and may place the biological sample onto the surface of the conveyor 700 within the chamber 106. The first door 107 of the chamber 106 may close, while the second door 108 remains closed. In some embodiments, the exterior of the biological sample, the first robotic arm 201, and/or the second robotic arm 202 may be sanitized and/or sterilized within the chamber 106. The conveyor 700 within the chamber 106 may transport (e.g., convey) the biological sample from a location proximate the first section 101 to a location proximate the second section 102 of the laboratory 100. The second door 108 may open, while the first door 107 remains closed. At least a portion of the second robotic arm 202 in the second section 102 of the laboratory 100 may extend through the open second door 108 into the chamber 106, and the second robotic arm 202 may pick up the biological sample from the surface of the conveyor 700 within the chamber 106. The second robotic arm 202 may transport the biological sample from the chamber 106 to the second section 102, and put down the biological sample in the second section 102. The second robotic arm 202 may return to the second section 102 of the laboratory 100, such that the second robotic arm 202 is fully within the second section 102. If the second robotic arm 202 is located within the chamber 106, the second robotic arm 202 may pick up the biological sample from the surface of the conveyor 700, the second door 108 may open, and the second robotic arm 202 may extend through the open second door 108 and place the biological sample in the second section 102 of the laboratory 100. The second robotic arm 202 may then withdraw through the second door 108 back into the chamber 106. In some examples, the second robotic arm 202 may place the biological sample on or within reach of the second conveyance platform 112. The second door 108 may close, while the first door 107 remains closed. In some embodiments, the chamber 106 and/or any component within the chamber 106 may be sanitized and/or sterilized at any point during, before, and/or after transportation of the biological sample from the first section 101 to the second section 102 of the laboratory 100.

In some embodiments, to transport the biological sample from the second section 102 to the first section 101 of the laboratory 100, the second robotic arm 202 in the second section 102 may pick up the biological sample in the second section 102. In some examples, the second robotic arm 202 may pick up the biological sample from the second conveyance platform 112. The second door 108 of the chamber 106 may open while the first door 107 remains closed, such that the second section 102 of the laboratory 100 is isolated from the first section 101. At least a portion of the second robotic arm 202 may extend through the open second door 108 into the chamber 106, transporting the biological sample from the second section 102 to the chamber 106, and the second robotic arm 202 may place the biological sample onto the surface of the conveyor 700 within the chamber 106. The second robotic arm 202 may return to the second section 102 of the laboratory 100, such that the second robotic arm 202 is fully within the second section 102. If the second robotic arm 202 is located within the chamber 106, the second door 108 may open, the second robotic arm 202 may extend through the open second door 108, and the second robotic arm 202 may pick up the biological sample in the second section 102 of the laboratory 100. The second robotic arm 202 may then withdraw through the second door 108 back into the chamber 106 and may place the biological sample onto the surface of the conveyor 700 within the chamber 106. The second door 108 of the chamber 106 may close, while the first door 107 remains closed. In some embodiments, the exterior of the biological sample, the first robotic arm 201, and/or the second robotic arm 202 may be sanitized and/or sterilized within the chamber 106. The conveyor 700 within the chamber 106 may transport the biological sample from the location proximate the second section 102 of the laboratory 100 to the location proximate the first section 101. The first door 107 may open, while the second door 108 remains closed. At least a portion of the first robotic arm 201 in the first section 101 may extend through the open first door 107 into the chamber 106, and the first robotic arm 201 may pick up the biological sample from the surface of the conveyor 700 within the chamber 106. The first robotic arm 201 may transport the biological sample from the chamber 106 to the first section 101, and put down the biological sample in the first section 101. The first robotic arm 201 may return to the first section 101 of the laboratory 100, such that the first robotic arm 201 is fully within the first section 101. If the first robotic arm 201 is located within the chamber 106, the first robotic arm 201 may pick up the biological sample from the surface of the conveyor 700, the first door 107 may open, and the first robotic arm 201 may extend through the open first door 107 and place the biological sample in the first section 101 of the laboratory 100. The first robotic arm 201 may then withdraw through the first door 107 back into the chamber 106. In some examples, the first robotic arm 201 may place the biological sample on or within reach of the first conveyance platform 111. The first door 107 may close, while the second door 108 remains closed. In some embodiments, the chamber 106 and/or any component within the chamber 106 may be sanitized and/or sterilized at any point during, before, and/or after transportation of the biological sample from the second section 102 to the first section 101 of the laboratory 100.

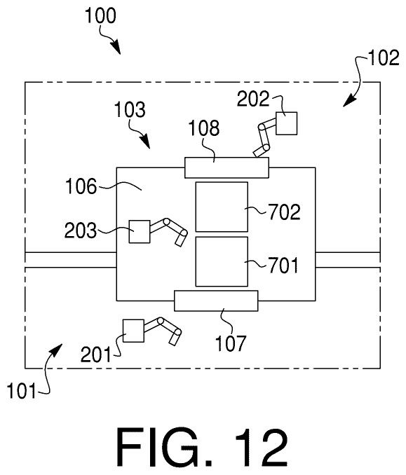

FIG. 12 depicts a schematic top view of the portion of the laboratory of FIG. 1, with the physical separation system including three robotic arms and two conveyors, according to one or more embodiments of the present disclosure. As FIG. 12 illustrates, the physical separation system 103 may include the first robotic arm 201 in the first section 101 of the laboratory 100, the second robotic arm 202 in the second section 102 of the laboratory 100, and a third robotic arm 203 in the chamber 106. The physical separation system 103 may further include a first conveyor 701 and a second conveyor 702 within the chamber 106. In some aspects, the first robotic arm 201 and the second robotic arm 202 may be located in the chamber 106 with the third robotic arm 203. In some embodiments, the first robotic arm 201 may transport the biological sample between the first section 101 of the laboratory 100 and the first conveyor 701, the second robotic arm 202 may transport the biological sample between the second conveyor 702 and the second section 102 of the laboratory 100, and the third robotic arm 203 may transport the biological sample between the first conveyor 701 and the second conveyor 702 within the chamber 106. The first robotic arm 201 may pick up or place the biological sample from, on, or within reach of the first conveyance platform 111, and the second robotic arm 202 may pick up or place the biological sample from, on, or within reach of the second conveyance platform 112.

In some embodiments, to transport the biological sample from the first section 101 to the second section 102 of the laboratory 100, the first robotic arm 201 in the first section 101 may pick up the biological sample in the first section 101. In some examples, the first robotic arm 201 may pick up the biological sample from the first conveyance platform 111. The first door 107 of the chamber 106 may open while the second door 108 remains closed, such that the first section 101 of the laboratory 100 is isolated from the second section 102. At least a portion of the first robotic arm 201 may extend through the open first door 107 into the chamber 106, transporting the biological sample from the first section 101 to the chamber 106. The first robotic arm 201 may place the biological sample onto the first conveyor 701 within the chamber 106. The first robotic arm 201 may return to the first section 101 of the laboratory 100, such that the first robotic arm 201 is fully within the first section 101. If the first robotic arm 201 is located within the chamber 106, the first door 107 may open, the first robotic arm 201 may extend through the open first door 107, and the first robotic arm 201 may pick up the biological sample in the first section 101 of the laboratory 100. The first robotic arm 201 may then withdraw through the first door 107 back into the chamber 106 and may place the biological sample onto the surface of the first conveyor 701 within the chamber 106. The first door 107 may close, while the second door 108 remains closed. In some embodiments, the exterior of the biological sample, the first robotic arm 201, and/or the second robotic arm 202 may be sanitized and/or sterilized within the chamber 106. The first conveyor 701 within the chamber 106 may transport the biological sample from a location proximate the first section 101 to a location proximate the second conveyor 702. The third robotic arm 203 may pick up the biological sample from the first conveyor 701 within the chamber 106, and place the biological sample onto the second conveyor 702 within the chamber 106. The second conveyor 702 within the chamber 106 may transport the biological sample from the location proximate the first conveyor 701 to a location proximate the second section 102 of the laboratory 100. The second door 108 of the chamber 106 may open, while the first door 107 remains closed. At least a portion of the second robotic arm 202 in the second section 102 may extend through the open second door 108 into the chamber 106, and the second robotic arm 202 may pick up the biological sample from the second conveyor 702 within the chamber 106. The second robotic arm 202 may transport the biological sample from the chamber 106 to the second section 102, and put down the biological sample in the second section 102. The second robotic arm 202 may return to the second section 102 of the laboratory 100, such that the second robotic arm 202 is fully within the second section 102. If the second robotic arm 202 is located within the chamber 106, the second robotic arm 202 may pick up the biological sample from the surface of the second conveyor 702, the second door 108 may open, and the second robotic arm 202 may extend through the open second door 108 and place the biological sample in the second section 102 of the laboratory 100. The second robotic arm 202 may then withdraw through the second door 108 back into the chamber 106. In some examples, the second robotic arm 202 may place the biological sample on or within reach of the second conveyance platform 112. The second door 108 may close, while the first door 107 remains closed. In some embodiments, the chamber 106 and/or any component within the chamber 106 may be sanitized and/or sterilized at any point during, before, and/or after transportation of the biological sample from the first section 101 to the second section 102 of the laboratory 100.

In some embodiments, to transport the biological sample from the second section 102 to the first section 101 of the laboratory 100, the second robotic arm 202 in the second section 102 may pick up the biological sample in the second section 102. In some examples, the second robotic arm 202 may pick up the biological sample from the second conveyance platform 112. The second door 108 of the chamber 106 may open while the first door 107 remains closed, such that the second section 102 of the laboratory 100 is isolated from the first section 101. At least a portion of the second robotic arm 202 may extend through the open second door 108 into the chamber 106, transporting the biological sample from the second section 102 to the chamber 106, and the second robotic arm 202 may place the biological sample onto the second conveyor 702 within the chamber 106. The second robotic arm 202 may return to the second section 102 of the laboratory 100, such that the second robotic arm 202 is fully within the second section 102. If the second robotic arm 202 is located within the chamber 106, the second door 108 may open, the second robotic arm 202 may extend through the open second door 108, and the second robotic arm 202 may pick up the biological sample in the second section 102 of the laboratory 100. The second robotic arm 202 may then withdraw through the second door 108 back into the chamber 106 and may place the biological sample onto the surface of the second conveyor 702 within the chamber 106. The second door 108 of the chamber 106 may close, while the first door 107 remains closed. In some embodiments, the exterior of the biological sample, the first robotic arm 201, and/or the second robotic arm 202 may be sanitized and/or sterilized within the chamber 106. The second conveyor 702 within the chamber 106 may transport the biological sample from the location proximate the second section 102 to the location proximate the first conveyor 701. The third robotic arm 203 may pick up the biological sample from the second conveyor 702 within the chamber 106, and place the biological sample onto the first conveyor 701 within the chamber 106. The first conveyor 701 within the chamber 106 may transport the biological sample from the location proximate the second conveyor 702 to the location proximate the first section 101 of the laboratory 100. The first door 107 of the chamber 106 may open, while the second door 108 remains closed. At least a portion of the first robotic arm 201 in the first section 101 may extend through the open first door 107 into the chamber 106, and the first robotic arm 201 may pick up the biological sample from the first conveyor 701 within the chamber 106. The first robotic arm 201 may transport the biological sample from the chamber 106 to the first section 101, and put down the biological sample in the first section 101. The first robotic arm 201 may return to the first section 101 of the laboratory 100, such that the first robotic arm 201 is fully within the first section 101. If the first robotic arm 201 is located within the chamber 106, the first robotic arm 201 may pick up the biological sample from the surface of the first conveyor 701, the first door 107 may open, and the first robotic arm 201 may extend through the open first door 107 and place the biological sample in the first section 101 of the laboratory 100. The first robotic arm 201 may then withdraw through the first door 107 back into the chamber 106. In some examples, the first robotic arm 201 may place the biological sample on or within reach of the first conveyance platform 111. The first door 107 may close, while the second door 108 remains closed. In some embodiments, the chamber 106 and/or any component within the chamber 106 may be sanitized and/or sterilized at any point during, before, and/or after transportation of the biological sample from the second section 102 to the first section 101 of the laboratory 100.

Embodiments of the disclosure described herein may include one robotic arm, two robotic arms, or three or more robotic arms, and may include one or more conveyance systems (e.g., carousels, turntables, conveyors, drawers, rails, etc.) within the chamber 106. If one robotic arm is used, then the robotic arm may extend into both the first section 101 and the second section 102 of the laboratory 100. While this may be satisfactory to avoid cross-contamination in some aspects, the use of more robotic arms may further inhibit cross-contamination. For example, the use of two robotic arms may mean that each robotic arm only operates within and is exposed to one section of the laboratory 100. Further, if three robotic arms are used, then an additional buffer is created between a robotic arm that operates within and is exposed to one section of laboratory 100 and a robotic arm that operates within and is exposed to another section of laboratory 100. In such embodiments in which three robotic arms are included, no arm that operates within and is exposed to a section of the laboratory 100 may come into contact with or directly pass a biological sample to another arm that operates within and is exposed to another section of the laboratory 100. Instead, an intermediate third arm may be used to pass the biological sample between the two robotic arms. The use of a conveyance system (e.g., carousels, turntables, conveyors, drawers, rails, etc.) may also act as a buffer between or among the robotic arms. Thus use of multiple conveyance systems (e.g., carousels, turntables, conveyors, drawers, rails, etc.), may further provide a buffer so that each conveyance system is only ever exposed to one side of the laboratory 100, at most.

Although the transport of a sample is described for each embodiment in a bidirectional manner, i.e., describing transport both from the first section 101 to the second section 102, and from the second section 102 to the first section 101, in some processes, the sample may only ever be transported in one direction, e.g., either from first section 101 to second section 102, or from the second section 102 to the first section 101.

Further, while the Specification describes the movement of a sample for convenience, “a sample” may refer to one sample or to a plurality of samples, e.g., a batch of samples or a container in which a plurality of samples are located. Although description of “a sample” is included in reference to FIGS. 1-12, a plurality of samples may be loaded and unloaded by the one or more robotic arms, and/or may be loaded and unloaded onto a carousel, turntable, drawer, pneumatic carrier or tube, conveyor, etc. As one example, the first robotic arm may pick up and load a plurality of samples into the same opening on a carousel or into multiple openings on a carousel before the carousel is rotated to the next position. Likewise, the second robotic arm may pick up and unload a plurality of samples from the same opening on the carousel or from multiple openings on the carousel. In some aspects, the carousel, the conveyor, the turntable, or other mechanisms described herein may be used incrementally, e.g., with the first robotic arm loading a sample onto the carousel, the carousel turning less than 180 degrees to expose an empty opening, and then the first robotic arm loading another sample onto the carousel, and, in another section of the laboratory, the second robotic arm may be unloading samples from the carousel as the carousel turns incrementally. In this aspect, the last sample moved onto the carousel by the first robotic arm may not be the next sample unloaded by the second robotic arm. This may increase the throughput of samples transferred between or among different sections of the laboratory. This may apply to each of the mechanisms and embodiments described in FIGS. 2-12.

Additionally, as described above, although the embodiments refer to biological samples, the samples described in reference to FIGS. 1-12 may be any suitable type of sample that would be transported in any industry. And, although reference is made to laboratory 100, laboratory 100 may be used in any suitable industry, not only laboratories for biological samples. In one example, a system that incorporates the innovative concepts described herein may be well suited, for example, for sample preparation and analysis in cancer detection analysis, where the ability to inhibit cross-contamination and the ability to automate sample transport may reduce the potential for human error, may enhance the ability of systems to handle large quantities of samples efficiency, and may enhance the reliability and accuracy of downstream cancer detection assays while maintaining or even improving long-term costs. For instance, the system may be capable of efficiently processing a large quantity of samples to extract nucleic acids and other biomarkers important for cancer diagnosis.

While one physical separation system 103 between the first section 101 and the second section 102 of the laboratory 100 is described herein, there may be a plurality of physical separation systems 103 between the first section 101 and the second section 102 of the laboratory 100. In some aspects, if multiple different sections of the laboratory 100 are separated from one another, then there may be one or more physical separation systems 103 between each of the laboratory sections (e.g., between third, fourth, etc., sections). Including multiple physical separation systems 103 between sections may inhibit or avoid bottlenecks resulting from transport of the samples between sections, and may increase sample throughput in the processing being performed.

In the embodiments described above with reference to FIGS. 2-12, the components responsible for moving the samples in physical separation systems 103 are described in detail. That said, other components may also be included within the physical separation systems 103 of FIGS. 2-12. For example, one or more identification (ID) scanners, such as a one-dimensional or two-dimensional barcode (e.g., QR code) scanner may be included within the physical separation systems 103 described herein. In this way, the identification of the sample being transferred by the physical separation systems 103 may be recorded and/or tracked, and/or movement through the laboratory 100 may be recorded and/or tracked. In some aspects, an ID scanner may be located in the first section 101 of the laboratory 100, in the second section 102 of the laboratory 100, and/or in the chamber 106. One or more of the first, second, and/or third robotic arms 201, 202, 203 may position the sample relative to an ID scanner to permit the ID scanner to scan an identifying label on the sample during the transfer processes described above with reference to FIGS. 2-12.

Incorporation of Embodiments of the Disclosure into an Exemplary Automated System The novel concepts described herein may be used in conjunction with an automated laboratory system, as is depicted in FIG. 1, which is managed by a novel computer system designed to achieve complete autonomy in laboratory workflows by leveraging a hierarchical software architecture, in which each layer of software in the hierarchical architecture is purposefully left generally ignorant, agnostic, and/or at least partially independent of the other layers. This novel system includes multiple independent software layers that distribute computational tasks at different layers of abstraction, thereby reducing data complexity and preventing computational overload. At the highest level, the top software layer orchestrates the overall workflow, managing the flow of samples and materials between or among various stations that perform various functions in an overall process, referred to herein as “work cells,” based on context data such as assay protocols, system component status, and inventory levels. The top software layer operates without awareness of the details of operations in the various work cells. The second software layer, functioning as a scheduler, manages tasks within individual work cells by mapping required work to available resources, allowing for the simultaneous processing of multiple sample batches. The second software layer operates without awareness of the other work cells or the broader workflow context. The third software layer controls specific instruments within the work cells, executing discrete operations without awareness of the other instruments in the work cell or the broader workflow context.

The innovative architecture described above ensures that each software layer operates independently and agnostically, enhancing the system's reliability and scalability. Additionally, the system's modular design allows for the addition or removal of hardware components, such as work cells and instruments, without necessitating system-wide shutdowns, thus maximizing uptime and operational efficiency. Moreover, the novel system may include an integrated information repository, which facilitates communication between layers, storing progress and quality assurance data and enabling the top software layer to monitor the overall health and workflow status of the system. Accordingly, by eliminating the need for human intervention in work allocation, sample handling, and movement, this fully autonomous system reduces the risk of human error, thereby enhancing efficiency and enabling the processing of large numbers of samples (e.g., millions of samples) with precision and speed. Such a system is described in U.S. Provisional Patent Application No. 63/729,697, filed Dec. 9, 2024.

By dividing tasks among multiple independent software layers and purposefully keeping the software layers agnostic to one another, the system reduces the computational burden on any single layer. Each layer is responsible for specific functions, allowing for more efficient processing and avoiding the risk of system overload. This distribution enhances the system's reliability and performance, preventing the computational bottlenecks that commonly plague centralized systems.

Additionally, the system's architecture supports modifications, repairs, and updates while remaining operational (“online”). This “online” maintenance capability promotes continuous operation and minimal disruption, a significant improvement over conventional systems that require complete shutdowns for even maintenance tasks. In an aspect, dynamic task reallocation allows the top software layer to dynamically reassign work to alternative work cells or instruments if a specific component needs to be temporarily taken offline for servicing. Furthermore, the modular design of the novel system allows it to easily scale by the addition or removal of work cells and instruments as needed, without significantly compromising performance. This scalability is achieved without the need for extensive reconfiguration or system downtime, making it possible to adapt to varying throughput requirements and evolving laboratory needs. The ability to integrate additional hardware components ensures that the system can grow in response to increasing demand or new technological developments or shrink in response to decreased throughput needs or upstream efficiencies. Furthermore, the system is configured to autonomously manage complex workflows, including the movement and processing of samples, with precision and speed. This autonomy enables the system to process up to millions of samples accurately, improving throughput and reliability.

Although the physical separation systems 103 are described in this section as being incorporated into a specific, fully-automated laboratory system, such as the laboratory 100, embodiments of the disclosure may be incorporated into manual laboratory systems, semi-automated laboratory systems, or other types of automated laboratory systems using different types of system architectures than the exemplary system architecture described herein.

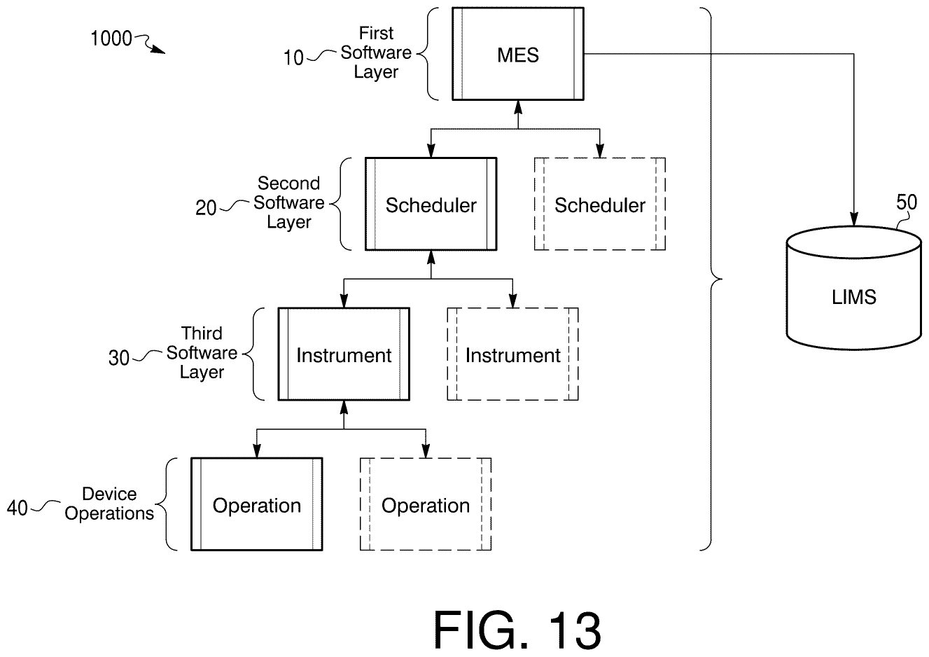

FIG. 13 depicts a block diagram illustrating a hierarchical software architecture 1000 for a fully automated system, which may incorporate embodiments of the disclosure. The software architecture 1000 may contain a plurality, e.g., three, software layers, including the top or first software layer 10, the second software layer 20, and the third software layer 30, with each layer responsible for its own specific set of data. The third software layer 30, which corresponds to instrument control within a work cell, may be responsible for executing specific device operations 40, as further discussed herein. In an aspect, the three software layers 10, 20, 30 may be in the form of software components that are stored on a memory and/or computer readable medium and that work with one or more processors (e.g., data processors) residing on one or more computer apparatuses. For example, all three software layers 10, 20, 30 may reside on a computer readable medium on one computational apparatus with one or more processors (e.g., microprocessors). Alternatively, the three software layers 10, 20, 30 may reside on three computer readable media residing on three separate computational apparatuses, each with one or more processors (e.g., microprocessors). In some aspects, the top software layer 10 may reside in a first computational apparatus (e.g., a first server computer), the second software layer 20 may reside in a second computational apparatus (e.g., a second server computer), and the third software layer 30 may reside in a third computational apparatus (e.g., a third server computer). Alternatively, a subset of the three software layers 10, 20, 30 may reside on two computer readable media residing on two separate computational apparatuses, each with one or more processors (e.g., microprocessors), such that two software layers reside on one computer readable media and one software layer resides on its own computer readable media.

The Laboratory Information Management System (LIMS) 50 is a component within the hierarchical software architecture 1000. Serving as a centralized information repository, LIMS 50 stores data generated throughout the workflow process. More particularly, LIMS 50 functions to provide a comprehensive database where relevant information about samples, processes, and system status is recorded. This includes data on sample identity, processing steps, reagent usage, and the status of various system components. By maintaining a centralized repository of such information, LIMS 50 enables the system to track the progress of each sample in real time, ensuring that all actions are accurately documented and traceable. This traceability promotes the integrity of the workflow and compliance with regulatory standards. In an aspect, LIMS 50 interfaces with each of the three software layers 10, 20, 30, facilitating the flow of information across the software architecture 1000. One or more software layers 10, 20, 30 may publish information to LIMS 50 while interfacing with samples or while awaiting samples. As further discussed herein, the top software layer 10 may access LIMS 50 to obtain context data, such as inventory levels, sample positions in the workflow process, instrument and component health indications, and the like, which are necessary for high-level decision-making and workflow orchestration. It is important to note that although FIG. 13 and the balance of this disclosure discusses a software architecture that contains three software layers, such a designation is not limiting. Specifically, fewer or additional software layers may be utilized in a system to facilitate the concepts described herein. For instance, instruments within the system may also be configured to oversee specific sub processes independently. This capability may allow individual instruments to manage tasks such as calibration, quality control checks, and data validation.

The fully automated system for which the hierarchical software architecture 1000 is constructed may include a variety of different hardware components. Provided below are descriptions of hardware components that may be utilized in the fully automated system. However, it is important to note that the number and type of hardware components may vary between systems and may be tailored to the overall goal of the fully automated system. Additional detail regarding how these hardware components interact and/or are controlled by the various software layers 10, 20, 30 are further provided herein.

The fully automated system may include one or more work cells which, in general, refer to a cluster of instruments that are each designed to execute various tasks in the furtherance of specific steps in the process workflow. More particularly, each work cell may be equipped with a variety of instruments and components tailored to the specific sample processing step(s) it was organized to perform. Some or all work cells may include a robotic component, such as one or more of the robotic arms described herein with reference to FIGS. 2-12 or another robotic arm, which may be linked to and controlled by a second software layer responsible for coordinating and executing tasks within the work cell. For instance, as an example, a pre-amplification processing work cell may be designed and equipped to handle various preparatory actions required for biological sample processing. It may contain a robotic arm capable of performing actions such as pipetting, mixing, and transferring samples between different instruments. In other embodiments, the robotic arm may pass biological samples between or among different instruments that each handle discrete tasks, such as pipetting, mixing, heating, cooling, etc. This robotic arm may ensure that each step, task, and/or operation is executed accurately, reducing the risk of human error. The physical separation systems 103 described herein may itself be considered a work cell, and may include at least one or more of, e.g., robotic arms, carousels, conveyors, turntables, drawers, pneumatic tubes, etc., as shown and described. In an aspect, the physical separation systems 103 may further include one or more ID scanners, such as a one-dimensional or two-dimensional barcode (e.g., QR code) scanner (e.g., which may be utilized to ensure identification of the sample being transferred, data logging, etc.), and the like. As another example, another type of work cell may be a post-amplification processing work cell, which may be designed to handle tasks related to the amplification and subsequent processing of biological samples. Such a work cell may include a variety of equipment, some or all of which may be different from the pre-amplification work cell, including: a polymerase chain reaction (PCR) machine (e.g., which is utilized for amplifying DNA samples through PCR, thereby increasing the quantity of DNA), a thermocycler (e.g., which may be utilized to facilitate the cycling of temperatures necessary for the denaturation, annealing, and extension phases of PCR), an automated gel electrophoresis system (e.g., which may be utilized for separating and analyzing DNA fragments, providing a means to verify the success and quality of the amplification process), and/or other devices not explicitly listed here.

Embodiments of the disclosure were designed to be ideal for use with such an automated system to solve a problem that had not been recognized previously in traditional laboratory environments in which only aspects of a process, and not a full start-to-finish process, had been automated. In traditional laboratory settings, human intervention to transport samples between segregated sections of a laboratory was acceptable, as human intervention was needed for other processing aspects, too. However, largescale processing and high throughput of samples, on the order, e.g., of up to millions of samples per year, may require full automation. The physical separation systems described herein may facilitate the full automation of processing, e.g., in a scenario of biological samples, the full automation of pre-amplification and post-amplification sample processing, while maintaining protocols to inhibit cross-contamination of pre-and post-amplification samples.

In the hierarchical software architecture described above, the top software layer may control movement of samples to the physical separation system once all of the processing steps in the first laboratory section are complete and the sample is ready to be transferred to the second laboratory section for further processing. The second software layer may control the transfer of the sample within the physical separation system from the first laboratory section to the second laboratory section. The third software layer may control the specific instruments within the physical separation system (e.g., the robotic arm(s), the doors, the conveyance mechanisms (e.g., one or more carousels, turntables, drawers, conveyors, pneumatic systems, sterilization equipment, etc.)).

If multiple physical separation systems are included between the first laboratory section and the second laboratory section, then the first layer of software may determine which physical separation system has the capacity to accept a sample, and thus which physical separation system to route the sample to. If one or more physical separation systems are offline, are operating at reduced capacity, or are full, then the first layer of software may route fewer samples to that physical separation system, or may route samples to another physical separation system able to transport the sample in a more timely manner. This may alleviate or inhibit bottlenecks that may otherwise occur when transferring samples from the first laboratory section to the second laboratory section using the physical separation system.

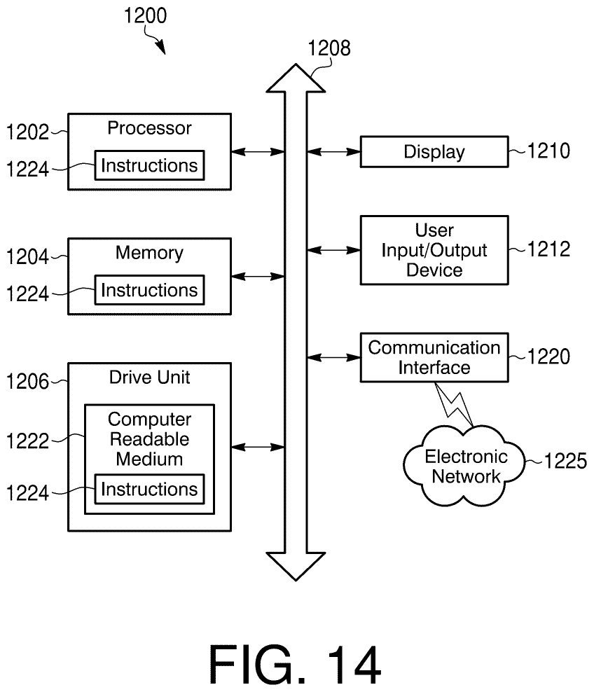

FIG. 14 is a simplified functional block diagram of a computer system 1200 that may be configured as a computing device for executing the processes described herein, according to exemplary embodiments of the present disclosure. In various embodiments, any of the systems herein may be an assembly of hardware including, for example, a data communication interface 1220 for packet data communication. The platform also may include a central processing unit (“CPU”) or processor 1202, in the form of one or more processors, for executing program instructions. The platform may include an internal communication bus 1208, and a storage or drive unit 1206 (such as ROM, HDD, SDD, etc.) that may store data on a computer readable medium 1222, although the computer system 1200 may receive programming and data via network communications via electronic network 1225 (e.g., voice, video, audio, images, or any other data over the electronic network 1225). The computer system 1200 may also have a memory 1204 (such as RAM) storing instructions 1224 for executing techniques presented herein, although the instructions 1224 may be stored temporarily or permanently within other modules of the computer system 1200 (e.g., the processor 1202 and/or computer readable medium 1222). The computer system 1200 also may include user input and output ports 1212 and/or a display 1210 to connect with input and output devices such as keyboards, a mouse or mice, touchscreens, monitors, displays, etc. The various system functions may be implemented in a distributed fashion on a number of similar platforms, to distribute the processing load. Alternatively, the systems may be implemented by appropriate programming of one computer hardware platform.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of ±10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only if the alternatives are mutually exclusive, although the disclosure supports a definition that refers to only alternatives and “and/or.” As used herein “another” may mean at least a second or more.

As used herein, the term “user” generally encompasses any person or entity, such as a laboratory technician, researcher and/or a care provider (e.g., a doctor, etc.), who may desire information, resolution of an issue, or engage in any other type of interaction with a provider of the systems and methods described herein (e.g., via an application interface resident on their electronic device, etc.). The term “electronic application” or “application” may be used interchangeably with other terms like “program,” or the like, and generally encompasses software that is configured to interact with, modify, override, supplement, or operate in conjunction with other software.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present disclosure.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

1. A physical separation system configured to transport a sample between a first section of a laboratory configured to perform a first portion of a process on the sample, and a second section of the laboratory configured to perform a second portion of the process on the sample, the system comprising:
a chamber,
a first door positionable to optionally permit or prevent access between the first section and the chamber,
a second door positionable to optionally permit or prevent access between the second section and the chamber, and
a first robotic arm configured to transport the sample between at least one of:
the first section and the chamber,
the second section and the chamber, or
a first location within the chamber and a second location within the chamber.

2. The system of claim 1, wherein at least one of the first section or the second section includes equipment configured to perform pre-amplification of nucleic acids within the sample.

3. The system of claim 2, wherein another of the first section or the second section includes equipment configured to perform post-amplification of nucleic acids within the sample.

4. The system of claim 1, wherein the first robotic arm is located within the chamber and is configured to transport the sample between the first section and the chamber, and between the second section and the chamber.

5. The system of claim 1, wherein the sample is a biological sample.

6. The system of claim 1, wherein the chamber is disposed within a wall of the laboratory.

7. The system of claim 1, wherein the first robotic arm is disposed within one of the first section or the chamber, and
wherein the physical separation system further comprises:
a second robotic arm disposed within one of the second section or the chamber, and
a third robotic arm disposed within the chamber,
wherein the first robotic arm is configured to transport the sample between the first section and the chamber,
wherein the second robotic arm is configured to transport the sample between the second section and the chamber, and
wherein the third robotic arm is configured to transport the sample between a first location within the chamber and a second location within the chamber.

8. The system of claim 1, wherein the first robotic arm is disposed within one of the first section or the chamber, and
wherein the physical separation system further comprises:
a second robotic arm disposed within one of the second section or the chamber,
a third robotic arm disposed within the chamber,
a first carousel disposed within the chamber, the first carousel comprising at least a first opening configured to receive the sample, and
a second carousel disposed within the chamber, the second carousel comprising at least a second opening configured to receive the sample,
wherein the first robotic arm is configured to transport the sample between the first section and the first opening of the first carousel,
wherein the second robotic arm is configured to transport the sample between the second section and the second opening of the second carousel, and
wherein the third robotic arm is configured to transport the sample between the first opening of the first carousel and the second opening of the second carousel.

9. The system of claim 8, wherein the first carousel is configured to rotate between a position in which the first opening is proximate the first door and a position in which the first opening is proximate the second carousel, and
wherein the second carousel is configured to rotate between a position in which the second opening is proximate the first carousel and a position in which the second opening is proximate the second door.

10. The system of claim 1, wherein the first robotic arm is disposed within one of the first section or the chamber, and
wherein the physical separation system further comprises:
a second robotic arm disposed within one of the second section or the chamber,
a third robotic arm disposed within the chamber,
a first turntable disposed within the chamber, the first turntable comprising at first surface configured to receive the sample, and
a second turntable within the chamber, the second turntable comprising a second surface configured to receive the sample,
wherein the first robotic arm is configured to transport the sample between the first section and the first surface of the first turntable,
wherein the second robotic arm is configured to transport the sample between the second section and the second surface of the second turntable, and
wherein the third robotic arm is configured to transport the sample between the first surface of the first turntable and the second surface of the second turntable.

11. The system of claim 10, wherein the first turntable is configured to rotate between a position in which a portion of the first turntable on which the sample is received is proximate the first door and a position in which the portion of the first turntable on which the sample is received is proximate the second turntable, and
wherein the second turntable is configured to rotate between a position in which a portion of the second turntable on which the sample is received is proximate the first turntable and a position in which the portion of the second turntable on which the sample is received is proximate the second door.

12. The system of claim 1, wherein the first robotic arm is disposed within one of the first section or the chamber, and
wherein the physical separation system further comprises:
a second robotic arm disposed within one of the second section or the chamber,
a third robotic arm disposed within the chamber,
a first conveyor within the chamber, the first conveyor comprising a first surface configured to receive the sample, and
a second conveyor within the chamber, the second conveyor comprising a second surface configured to receive the sample,
wherein the first robotic arm is configured to transport the sample between the first section and the first surface of the first conveyor,
wherein the second robotic arm is configured to transport the sample between the second section and the second surface of the second conveyor, and
wherein the third robotic arm is configured to transport the sample between the first surface of the first conveyor and the second surface of the second conveyor.

13. The system of claim 12, wherein the first conveyor is configured to convey the sample between a position proximate the first door and a position proximate the second conveyor, and
wherein the second conveyor is configured to convey the sample between a position proximate the first conveyor and a position proximate the second door.

14. The system of claim 1, wherein the first robotic arm is disposed within one of the first section or the chamber, and
wherein the physical separation system further comprises:
a second robotic arm disposed within one of the second section or the chamber,
wherein the first robotic arm is configured to transport the sample between the first section and the chamber, and
wherein the second robotic arm is configured to transport the sample between the second section and the chamber.

15. The system of claim 1, wherein the first robotic arm is disposed within one of the first section or the chamber, and
wherein the physical separation system further comprises:
a second robotic arm disposed within one of the second section or the chamber, and
a carousel disposed within the chamber, the carousel comprising an opening configured to receive the sample, and
wherein the first robotic arm is configured to transport the sample between the first section and the opening of the carousel, and
wherein the second robotic arm is configured to transport the sample between the second section and the opening of the carousel.

16. The system of claim 15, wherein the carousel is configured to rotate the opening between a position proximate the first door and a position proximate the second door.

17. The system of claim 1, wherein the first robotic arm is disposed within one of the first section or the chamber, and
wherein the physical separation system further comprises:
a second robotic arm disposed within one of the second section or the chamber, and
a turntable disposed within the chamber, the turntable comprising a surface configured to receive the sample, and
wherein the first robotic arm is configured to transport the sample between the first section and the surface of the turntable, and
wherein the second robotic arm is configured to transport the sample between the second section and the surface of the turntable.

18. The system of claim 17, wherein the turntable is configured to rotate the sample between a position proximate the first door and a position proximate the second door.

19. The system of claim 1, wherein the first robotic arm is disposed within one of the first section or the chamber, and
wherein the physical separation system further comprises:
a second robotic arm disposed within one of the second section or the chamber, and
a conveyor disposed within the chamber, the conveyor comprising a surface configured to receive the sample, and
wherein the first robotic arm is configured to transport the sample between the first section and the surface of the conveyor, and
wherein the second robotic arm is configured to transport the sample between the second section and the surface of the conveyor.

20. The system of claim 19, wherein the conveyor is configured to convey the sample between a position proximate the first door and a position proximate the second door.

21.-54. (canceled)
